(19) United States
(12) Patent Application Publication
(10) Pub. No.: US 2010/0292309 A1
(43) Pub. Date: Nov. 18, 2010

(54) INDUCING IMMUNE-MEDIATED TUMOR CELL DEATH

(75) Inventors: Richard G. Vile, Rochester, MN (US); Timothy J. Kottke, Oronoco, MN (US); Jose S. Pulido, Rochester, MN (US); Jill M. Thompson, Stewartville, MN (US); Alan Melcher, Leeds (GB)

Correspondence Address: FISH & RICHARDSON P.C., PO BOX 1022, MINNEAPOLIS, MN, 55440-1022, US

(73) Assignee: MAYO FOUNDATION FOR MEDICAL EDUCATION AND RESEARCH

(21) Appl. No.: 12/530,290

(22) PCT Filed: Mar. 7, 2008
(86) PCT No.: PCT/US08/56223

§ 371 Date: Aug. 2, 2010

Related U.S. Application Data
(60) Provisional application No. 60/905,861, filed on Mar. 8, 2007.

(51) Int. Cl. A61K 31/7088; C07H 21/04; C12N 15/63; A61P 35/00

(52) U.S. Cl. 514/44 R; 536/23.5; 536/23.2; 435/320.1

(57) ABSTRACT

This document provides methods and materials related to treating cancer. For example, methods and materials that include using CD40L polypeptide, an hsp70 polypeptide, and a cytotoxic polypeptide to trigger an immune response directed against cancer cells are provided.

### CROSS-REFERENCE TO RELATED APPLICATIONS

This application claims priority from U.S. Provisional Application Ser. No. 60/905,861, filed Mar. 8, 2007, which is incorporated herein by reference in its entirety.

### STATEMENT AS TO FEDERALLY SPONSORED RESEARCH

This invention was made with government support under grant nos. CA085931, CA094180, and CA107082, awarded by National Institutes of Health. The government has certain rights in the invention.

### BACKGROUND

1. Technical Field

This document relates to methods and materials involved in killing tumor cells (e.g., melanoma cells).

2. Background Information

Most current strategies designed to generate immune-mediated responses to tumors involve the use of tumor-associated antigens. Effective strategies promote the release of tumor-associated antigens in the presence of potent inflammatory signals to induce T cell-mediated killing of tumor cells (Pardoll, Nat. Rev. Immunol., 2:227-38 (2002); Huang et al., Science, 264:961-5 (1995); Gallucci et al., Nat. Med., 5:1249-55 (1994); Melcher et al., J. Mol. Med., 77:824-33 (1999); Liu et al., J. Exp. Med., 196:1091-7 (2002); Mougneau et al., J. Exp. Med., 196:1013-6 (2002)). These strategies require time-consuming and expensive protocols to isolate tumor-derived materials (e.g., tumor-derived cells, tumor-derived cell lysates, tumor-derived proteins, or tumor-derived peptides).

### SUMMARY

This document provides methods and materials related to treating cancer. The methods and materials provided herein are based, in part, on the discovery that CD40 ligand (CD40L) polypeptides and chaperone polypeptides (e.g., hsp70 polypeptides) can be used together with a cytotoxic polypeptide to trigger an immune response directed against cancer cells.

In general, one aspect of this document features an isolated nucleic acid comprising, or consisting essentially of, (a) a sequence encoding a CD40L polypeptide, a sequence encoding a chaperone polypeptide, and a sequence encoding a cytotoxic polypeptide; (b) a sequence encoding a CD40L polypeptide and a sequence encoding chaperone polypeptide; or (c) a sequence encoding a CD40L polypeptide and a sequence encoding a cytotoxic polypeptide. The CD40L polypeptide can be a human CD40L polypeptide. The chaperone polypeptide can be a human hsp70 polypeptide. The cytotoxic polypeptide can be a herpes simplex virus thymidine kinase polypeptide or a fusogenic membrane G glycoprotein of vesicular stomatitis virus. The nucleic acid can be a plasmid. The nucleic acid can be a viral vector.

In another aspect, this document features a composition comprising, or consisting essentially of, (a) a nucleic acid molecule encoding a CD40L polypeptide, a nucleic acid molecule encoding a chaperone polypeptide, and a nucleic acid molecule encoding a cytotoxic polypeptide; (b) a nucleic acid molecule encoding a CD40L polypeptide and a nucleic acid molecule encoding a chaperone polypeptide; or (c) a nucleic acid molecule encoding a CD40L polypeptide or a nucleic acid molecule encoding a cytotoxic polypeptide. The CD40L polypeptide can be a human CD40L polypeptide. The chaperone polypeptide can be a human hsp70 polypeptide. The cytotoxic polypeptide can be a herpes simplex virus thymidine kinase polypeptide or a fusogenic membrane G glycoprotein of vesicular stomatitis virus. One or more of the nucleic acid molecules can be a plasmid. One or more of the nucleic acid molecules can be a viral vector.

In another aspect, this document features a method for inducing immunity against cancer. The method comprises, or consists essentially of, administering nucleic acid encoding a CD40L polypeptide, a chaperone polypeptide, and a cytotoxic polypeptide to a mammal having the cancer under conditions wherein the CD40L polypeptide, the chaperone polypeptide, and the cytotoxic polypeptide are expressed. The mammal can be a human. The cancer can be a melanoma cancer or a prostatic cancer. The nucleic acid can be a single nucleic acid encoding the CD40L polypeptide, the chaperone polypeptide, and the cytotoxic polypeptide.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1A is a graph plotting survival (tumor size exceeding 1.0 cm) for C57BL/6 mice that were seeded with B16 tumors s.c. on day 1, injected i.d. with Tyr-HSVtk+CMV-hsp70 or Tyr-HSVtk+CMV-LacZ plasmids on days 4, 5, 6, 11, 12, 13, 18, 19, and 20, and treated with ganciclovir (GCV) administered i.p. on days 4-8, 11-15, and 18-22. FIG. 1B is a graph plotting interferon-gamma (IFN-γ) levels in supernatants from splenocytes that were recovered from naive mice or from mice 5 days following the first of three daily i.d. injections of Tyr-HSVtk/CMV-hsp70 or Tyr-HSVtk/CMV-LacZ and 5 daily injections of GCV. Splenocytes from each treatment group were divided into four separate cultures and stimulated with either no added peptide (-ve) or with the synthetic, H-2 Kb-restricted peptides hgp100₂₅₋₃₃, KVPRNQDWL (gp100; SEQ ID NO:1), TRP-2₁₈₀₋₁₈₈ SVYDFFVWL (TRP-2; SEQ ID NO:2) or Ova SIINFEKL (ova; SEQ ID NO:3) at 500,000 splenocytes per well in triplicate. Error bars represent standard deviations, and results from two separate mice per plasmid treatment are shown. FIG. 1C is a graph plotting survival (tumor size exceeding 1.0 cm) for TLR-4−/− C57BL/10ScNJ mice that were seeded with B16 tumors s.c. on day 1, injected i.d. with Tyr-HSVtk+CMV-hsp70 or Tyr-HSVtk+CMV-LacZ plasmids on days 4, 5, 6, 11, 12, 13, 18, 19, and 20, and treated with GCV administered i.p. on days 4-8, 11-15, and 18-22.

FIG. 2A is a picture of a gel showing TNF-α PCR products that were amplified using cDNA prepared from mouse skin samples that were taken at the site of three daily i.d. injections with Tyr-HSVtk+CMV-hsp70 or Tyr-HSVtk+CMV-LacZ (along with 5 injections i.p. of GCV). Skin samples were recovered four days following the first injection from three separate mice per plasmid combination. Glyceraldehyde-3-phosphate dehydrogenase (GAPDH) was used as a reference for levels of expression of each RNA. FIG. 2B is a picture of a gel as described for FIG. 2A, except that each group included two mice, using either C57Bl/6 mice or C57BL/10ScNJ (TLR 4−/−) mice for intradermal plasmid injections as indicated. GAPDH was used as a reference for levels of expression of each RNA. FIG. 2C is a graph plotting IFN-γ levels in splenocytes recovered from either C57BL/6 or TNF-α−/− mice 8 days following the first of three daily i.d. injections of Tyr-HSVtk/CMV-hsp70 or Tyr-HSVtk/CMV-LacZ and 5 daily injections of GCV. All groups of mice also received a single injection of Ad-LacZ or Ad-TNF-α along with the plasmid injections. IFN-γ ELISA results from splenocytes stimulated with the synthetic, H-2 Kb-restricted peptide TRP-2₁₈₀₋₁₈₈ SVYDFFVWL (TRP-2; SEQ ID NO:2) are shown.

FIGS. 3A-3D show the results of flow cytometry studies demonstrating that hsp70 expression induces trafficking of class II(Hi) cells to the draining lymph nodes (LN). A single cycle (three injections) of i.d. plasmid injections along with PBS or GCV for 5 consecutive days was administered to induce melanocyte killing, and cells were tracked from the site of injection to the draining LN by co-injecting the Cell Tracker Green (CTG) cell labeling dye along with the first plasmid injection. LN were analyzed six days later for CTG-labeled cells. Plasmid injection treatments are indicated for each panel, as are the % of CTG+ve, MHC Class II cells that reached the LN. The data shown are representative of multiple experiments.

FIG. 4A is a histogram plotting numbers of tetramer positive cells from draining inguinal LN in mice that received three daily i.d. injections of Tyr-HSVtk, pTyr-ova and CMV-hsp70, each given with GCV or PBS i.p. for 5 days. Cells were assayed by flow cytometry with SIINFEKL-(SEQ ID NO:3) loaded H-2 Kb Class I MHC tetramers for CD8+ T cells specific for the ova antigen. Tetramer positive cells (1.1%) were only detected in animals injected with Tyr-HSVtk/Tyr-ova/CMV-hsp70+GCV and no positive staining cells were detected using tetramers specific for TRP-2. FIG. 4B is a graph plotting the number of IFN-γ positive spots per well for LN cells from mice that were given a single cycle of i.d. injections of Tyr-HSVtk/CMV-hsp70/Tyr-ova or Tyr-HSVtk/CMV-LacZ/Tyr-ova, along with GCV. LN cells from three similarly injected animals were pooled, and CTG+ve/Class II+ve cells were recovered by FACS sorting. CTG+ve/Class II+ve cells were separated by flow cytometry sorting into CD11c+ or CD11c-ve populations, and portions of these cell populations were incubated directly with naive transgenic OT-1 T cells specific for the SIINFEKL (SEQ ID NO:3) epitope of ova and assayed after 72 hours by IFN-γ ELISPOT assays. Results shown are from a ratio of 100 OT-1:1 LN cell. The positive control, OT-1 incubated with SIINFEKL (SEQ ID NO:3) polypeptide gave >1000 spots per well under these conditions.

FIG. 5A is a picture of a gel showing PCR products generated using genomic DNA that was prepared from dissociated LN taken from mice subjected to a single cycle (three injections) of i.d. plasmid injections along with PBS or GCV for 5 consecutive days. DNA was screened for presence of the HSVtk transgene by PCR: Lane 1: Tyr-HSVtk (i.d.)+PBS (i.p); Lane 2: Tyr-HSVtk+CMV-hsp70+PBS; Lane 3: Tyr-HSVtk+CMV-hsp70+GCV; Lane 4: CMV-HSVtk+PBS; Lane 5: CMV-HSVtk+CMV-hsp70+PBS; Lane 6: CMV-HSVtk+CMV-hsp70+GCV; Lane 7: Tyr-HSVtk+GCV; Lane 8: CMV-HSVtk+GCV. Equal loading of DNA was confirmed by PCR for a 765 by genomic fragment of the tyrosinase promoter. Cumulative survival percentages, over three different experiments with 8-10 mice per group, using i.d. plasmid injections of Tyr-HSVtk/CMV-hsp70+GCV or CMV-HSVtk/CMV-hsp70+GCV are shown (p<0.0001). FIGS. 5B-5E are a series of plots showing trafficking of CTG labeled cells to the LN after i.d. plasmid injections. Plasmid injection treatments are indicated adjacent to each panel, as are the % of CTG+ve, MHC Class II cells that reached the LN. FIG. 5F is a picture of a gel showing levels of PCR products specific for the HSVtk transgene in mice subjected to a single cycle (three injections) of i.d. plasmid injections of Tyr-HSVtk/CMV-hsp70 (Lanes 1, 2, 5, 6) or Tyr-HSVtk/CMV-LacZ (lanes 3, 4, 7, 8), along with GCV for 5 consecutive days, which were administered to induce melanocyte killing in two mice per group in either C57BL/10ScNJ(TLR-4−/−) (lanes 1 and 2; 3 and 4) or C57Bl/6 mice (lanes 5 and 6 and 7 and 8). Six days after the first plasmid injection, LN were harvested and genomic DNA was screened for presence of the HSVtk transgene. Equal loading of DNA was confirmed by PCR for a 765 by genomic fragment of the tyrosinase promoter that is not within the injected plasmid. The data shown are representative of three separate experiments.

FIG. 6A is a table listing the percentage of long term (>60 days) survivor C57BL/6, or TLR-4−/− C57BL/10ScNJ mice that were seeded with B16 tumors s.c. on day 1, and injected i.d. on days 4, 5, 6, 11, 12, 13, 18, 19, and 20 with [Tyr-HSVtk+CMV-hsp70+CMV-LacZ] 10 μg each), [Tyr-HSVtk (10 μg)+CMV-LacZ (20 μg)], or [Tyr-HSVtk+CMV-hsp70+pCD40L] (10 μg each). GCV was administered i.p. on days 4-8, 11-15, and 18-22. The day at which the last mouse in each group was euthanized also is indicated. FIG. 6B is a diagram depicting the different treatment regimens used to treat small (3 day established) or larger (9 day established) disease in C57Bl/6 mice. The different regimens had different efficacies (p<0.005 between Tyr-HSVtk/CMV-hsp70 and Tyr-HSVtk/CMV-hsp70/pCD40L in the 9 day model of established disease). FIG. 6C is a graph plotting the number of spots appearing in IFN-γ coated ELISPOT wells that were seeded with 250,000 splenocytes per well in triplicate. The splenocytes were recovered from C57Bl/6 mice 9 days following the first of three daily i.d. injections of Tyr-HSVtk/CMV-hsp70/pCD40L, Tyr-HSVtk/CMV-hsp70 or Tyr-LacZ/CMV-hsp70 and 5 daily injections of GCV. Splenocytes from each treatment group were divided into three separate cultures and stimulated with either Ova SIINFEKL (ova; SEQ ID NO:3) (Control) or with the synthetic, H-2 Kb-restricted polypeptides hgp100₂₅₋₃₃, KVPRNQDWL (gp100; SEQ ID NO:1) or TRP-2₁₈₀₋₁₈₈ SVYDFFVWL (TRP-2; SEQ ID NO:2). Spot numbers were determined 72 hours after seeding. Error bars represent standard deviations. FIG. 6D is a table listing the mean activities of TRP-2 reactive cells in splenocytes of mice from different treatment groups, which was calculated from the total amount of IFN-γ detected by ELISA divided by the mean number of IFN-γ producing cells as determined by the ELISPOT analysis. Splenocytes were recovered from C57Bl/6 mice 9 days following the first of three daily i.d. plasmid injections as described for FIG. 6C, and were stimulated with the synthetic, H-2 Kb-restricted peptide TRP-2₁₈₀₋₁₈₈ SVYDFFVWL (TRP-2; SEQ ID NO:2) at 100,000 splenocytes per well in triplicate in IFN-γ ELISPOT wells. Supernatants were recovered after 72 hours and assayed for IFN-γ by ELISA. This experiment was repeated three times (p<0.02 for Tyr-HSVtk/CMV-hsp70/GCV compared to Tyr-HSVtk/CMV-hsp70/pCD40L/GCV). FIG. 6E is a picture of a gel showing PCR products generated using genomic DNA prepared from LN harvested from mice that received 3 daily i.d. injections of Tyr-HSVtk+/− CMV-hsp70+/−pCD40L, which were given with GCV i.p. for 5 days. LN were harvested after six days and genomic DNA assayed by PCR for HSVtk DNA. Equal loading of DNA was confirmed by PCR for a 765 by genomic fragment of the tyrosinase promoter. FIG. 6F is a histogram plotting numbers of ova-specific CD8+ T cells in LN harvested from mice that were treated with three daily i.d. injections of Tyr-HSVtk, Tyr-ova and CMV-hsp70 plasmids+/−pCD40L as indicated (given with GCV i.p. for 5 days). At day six, LN cells were assayed by FACS with SIINFEKL-(SEQ ID NO:3) loaded H-2 Kb class I MHC tetramers for ova-specific CD8+ T cells.

FIG. 7 is a graph plotting percent survival of C57BL/6 mice that were seeded with B16 tumors s.c. and subjected 3 rounds of i.d. plasmid treatment +GCV or PBS i.p. as indicated, starting on day 4. Groups A and B received Tyr-HSVtk/CMV-hsp70/empty plasmid; Group C received Tyr-HSVtk/CMV-hsp70/pCD40L. Group D (No tumor; pCD40L) received no initial tumor challenge but did receive Tyr-HSVtk/pCD40L/empty plasmid. Group B also received anti-CD40 Ab (FGK45 at 50 μg i.p. with each plasmid injection). In each group, 90 or 100% of the mice were cured of established tumors. On day 60, all survivors were re-challenged with 2×10⁵ B16 cells. Tumor size was monitored, and survival (tumor size exceeding 1.0 cm) following this re-challenge is shown.

FIG. 8 is a graph plotting mean weights of prostates from C57Bl/6 mice that were injected intraprostatically with Ad-GFP, Ad-VSV-G, Ad-hsp70, or Ad-VSV-G+Ad-hsp70. Mice were euthanized 45 days after intraprostatic injection of viruses.

FIG. 9A is a picture of a gel showing IL-6 and GAPDH PCR products generated using cDNA prepared from prostates of C57Bl/6 mice (two per group) that were injected intraprostatically with Ad-GFP, Ad-hsp70, or Ad-VSV-G, as indicated. Prostates were recovered three days after injection. FIG. 9B is a graph plotting IL-6 levels in supernatants of prostate cultures from three different C57Bl/6 mice. The cultures were incubated with recombinant murine hsp70 or bovine serum albumin (BSA) for 24 hours before IL-6 ELISA assays were done. Error bars represent the SD from three wells per sample in the ELISA assay. Results are representative of two separate experiments. FIG. 9C includes a graph plotting IL-6:GAPDH ratios and a picture of a gel showing IL-6 PCR products that were generated using cDNA prepared from draining LN of C57Bl/6 mice injected intraprostatically with Ad-GFP, Ad-VSV-G, or Ad-hsp70 or with Ad-VSV-G+Ad-hsp70. FIG. 9D is analogous to FIG. 9C, but includes a graph plotting TGF-β:GAPDH ratios and a gel showing TGF-β PCR products. Results in FIGS. 9C and 9D are presented as a ratio of the cytokine signal to the GAPDH signal for each treatment over at least three experiments. In addition, results in FIGS. 9A-9D are representative of multiple different experiments.

FIG. 10A is a graph plotting IL-17:GAPDH ratios in prostates of C57Bl/6 mice that were injected intraprostatically with Ad-GFP, Ad-VSV-G, Ad-hsp70, or Ad-VSV-G+Ad-hsp70, as indicated. After eight days, cDNA was prepared from the injected prostates, and PCR was used to analyze levels of IL-17 and GAPDH. Results are presented as a ratio of the cytokine signal to the GAPDH signal for each treatment over at least three experiments, and a sample gel is shown. FIG. 10B is a pair of graphs plotting the ratio of IL-17:GAPDH (top panel) using cDNA obtained from the experiment described for FIG. 9B (LN draining the prostates injected with adenoviral vectors). In addition, C57Bl/6 mice were treated intraprostatically with Ad-GFP, Ad-hsp70, Ad-VSV-G, or Ad-VSV-G+Ad-hsp70. After eight days, cells from the draining LN were cultured, and supernatants were assayed for IL-17 (bottom panel). *, positive with 30 additional cycles. FIG. 10C is a graph plotting IL-17 levels in splenocyte culture supernatants. C57Bl/6 mice were injected intraprostatically with no virus (lane 1) or with Ad-GFP (lane 2); i.d. with the Tyr-HSVtk/CMV-hsp70 plasmid combination shown previously to induce Treg cells (lane 3) or with Ad-VSV-G+Ad-hsp70 (lanes 4-6). Fourteen after injection of virus or plasmid (lanes 1-3, 7, 8), or 4 or 41 days postviral injection (lanes 4 and 6), splenocytes were recovered from treated mice and 250,000 were plated with 10⁵ naïve OT1 CD8⁺ T cells with H2K^{b}-restricted ova peptide SIINFEKL (SEQ ID NO:3) in triplicate. After 48 hours, supernatants were assayed for IF-γ. Lane 7, OT-1 with no added splenocytes; lane 8; OT-1 with the nonactivating TRP-2 peptide instead of SIINFEKL (SEQ ID NO:3). FIG. 10D is a graph plotting IFN-γ levels in supernatants from splenocyte cultures. C57Bl/6 mice were injected intraprostatically with plasmid expressing the cDNA for chick ovalbumin along with Ad-VSV-G or Ad-hsp70, or along with Ad-VSV-G+Ad-hsp70. All groups received an i.p. injection of either a control immunoglobulin (Ig) or the anti-CD25 Treg-depleting PC61 antibody 2 days before the intraprostatic plasmid/viral injection. After surgery, 500,000 splenocytes per treatment group were harvested and cocultured with the H-2K^{b} restricted ova peptide SIINFEKL (SEQ ID NO:3) in triplicate, and after 48 hours supernatants were assayed IFN-γ levels. Results shown are representative of two different experiments.

FIG. 11A is a graph plotting IFN-γ levels in supernatants from cultures of splenocytes isolated from C57Bl/6 (lanes 5-8) or B6.129S2-IL6^{tm1Kopf}/J (lanes 1-4) mice that were injected intraprostatically with Ad-GFP (lanes 3, 4, and 6) or with Ad-VSV-G+Ad-hsp70 (lanes 1, 2, and 5). On day 8, 250,000 splenocytes were plated with 10⁵ naïve OT-1 CD8⁺ T cells and H-2K^{b}-restricted ova peptide SIINFEKL (SEQ ID NO:3) in triplicate. After 48 hours, supernatants were assayed for IFN-γ. Lane 5, OT-1 with splenocytes from a C57Bl/6 mouse injected intraprostatically with Ad-VSV-G+Ad-hsp70; lane 6, OT-1 with splenocytes from a C57Bl/6 mouse injected with Ad-GFP in the prostate; lane 7, OT-1 with no added splenocytes; lane 8, splenocytes from a C57Bl/6 mouse with no added OT-1. FIG. 11B is a picture of a gel showing levels of IL-6, IL-17, and TGF-β PCR products using cDNA generated from prostates of C57Bl/6 (lanes 5 and 6) or B6.129S2-IL6^{tm1Kopf}/J (IL-6KO; lanes 1-4) mice that were injected intraprostatically with Ad-GFP (lanes 3, 4, and 6) or with Ad-VSV-G+Ad-hsp70 (lanes 1, 2, and 5). PCR for GAPDH showed equal loading.

FIG. 12A is a graph plotting percent survival for C57Bl/6 mice seeded s.c. with B16 or prostate TC2 cells. On day 6, mice were injected intraprostatically with Ad-GFP, Ad-VSV-G, Ad-hsp70, or Ad-VSV-G+Ad-hsp70. Survival (tumor, 1.0 cm) is shown for all TC2-bearing adenovirus-treated mice. Mice bearing B16 tumors s.c. treated with Ad-VSV-G+hsp70 intraprostatically also are shown. Results are representative of multiple experiments. FIG. 12B is a graph plotting IFN-γ levels in supernatants from cultures of splenocytes obtained from mice injected intraprostatically with Ad-GFP, Ad-VSV-G, Ad-hsp70, or Ad-VSV-G+Ad-hsp70. One week after surgery, 500,000 splenocytes per treatment group were harvested and cocultured with 50,000 TC2 or B 16 target tumor cells prepulsed with IFN-γ to increase levels of MHC class I. Forty-eight hours later, supernatants were harvested and assayed for IFN-γ. Cocultures of splenocytes with B16 targets produced no IFN-γ over background. Results are representative of two different experiments. FIG. 12C is a pair of graphs plotting the percent survival and percent tumor free mice after s.c. seeding with prostate TC2 cells and treatment as indicated. Top panel, prostate TC2 cells were seeded in C57BL/6 mice. On day four, mice received injections of control IgG or CD4⁺ T cell- or CD8⁺ T-cell-depleting antibodies. On day 6, mice were injected intraprostatically with Ad-VSV-G+Ad-hsp70. Survival (tumor, 1.0 cm) after seeding of tumors is shown. Results are representative of two different experiments. Bottom panel, prostate TC2 cells were seeded in B6.129S2-IL6^{tm1Kopf}/J (IL-6KO) mice. On day 6, mice were injected intraprostatically with Ad-GFP or with Ad-VSV-G+Ad-hsp70. FIG. 12D is a graph plotting IFN-γ levels for mice in FIG. 12C. When the B6.129S2-IL6^{tm1Kopf}/J (IL-6KO) mice were euthanized due to tumor size, 500,000 splenocytes per treatment group were harvested and cocultured with 50,000 TC2 or B 16 target tumor cells that were prepulsed with IFN-γ to increase levels of MHC class I. Splenocytes from a C57Bl/6 mouse injected intraprostatically with Ad-VSV-G+Ad-hsp70 were used as a positive control, as shown. After 48 hours, supernatants were harvested and assayed for IFN-γ. In addition, LN draining the injected prostates were recovered and assayed for IL-17 (LN IL-17). The only positive sample (>3 pg/mL) came from splenocytes from the C57Bl/6-treated mouse incubated with TC2 targets (35 pg/mL).

### DETAILED DESCRIPTION

This document provides methods and materials related to treating cancer (e.g., melanoma or prostate cancer). For example, this document provides methods and materials related to the use of a composition having nucleic acid encoding a cytotoxic polypeptide (e.g., a polypeptide encoded by a transcriptionally targeted cytotoxic gene), nucleic acid encoding a polypeptide having chaperone activity (e.g., heat shock protein (hsp70)), and nucleic acid encoding a polypeptide having CD40 ligand (CD40L) activity. Examples of polypeptides having chaperone activity include glycoprotein 96 (gp96), heat shock protein 90 (hsp90), heat shock protein 70 (hsp70), calreticulin, heat shock protein 110 (hsp110), heat shock protein 60 (hsp60), and glycoprotein 170 (gp 170). The cancer can comprise primary tumor cells or metastatic tumor cells. The cancer can be any type of cancer, including, without limitation, skin cancer (e.g., melanoma), prostate cancer, and breast cancer.

The terms “nucleic acid” and “polynucleotide” are used interchangeably herein, and refer to both RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

An “isolated” nucleic acid can be, for example, a naturally-occurring DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule, independent of other sequences (e.g., a chemically synthesized nucleic acid, or a cDNA or genomic DNA fragment produced by the polymerase chain reaction (PCR) or restriction endonuclease treatment). An isolated nucleic acid also refers to a DNA molecule that is incorporated into a vector, an autonomously replicating plasmid, or a virus. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

Isolated nucleic acid molecules can be produced by standard techniques. For example, polymerase chain reaction (PCR) techniques can be used to obtain an isolated nucleic acid containing a nucleotide sequence described herein. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid. Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, e.g., a naturally occurring DNA.

Vectors containing nucleic acids such as those described herein also are provided. A “vector” is a replicon, such as a plasmid, into which another DNA segment may be inserted so as to bring about the replication of the inserted segment. Generally, a vector is capable of replication when associated with the proper control elements. Suitable vector backbones include, for example, those routinely used in the art such as plasmids and viruses. The term “vector” includes cloning and expression vectors, as well as viral vectors and integrating vectors. An “expression vector” is a vector that includes a regulatory region. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

As used herein, a cytotoxic polypeptide or cytotoxic gene is a polypeptide or nucleic acid that, when expressed in a cell, causes the cell to die. Cell death can be by apoptosis or necrosis. A cytotoxic polypeptide or gene can cause a cell to die immediately upon expression or can require the presence of a prodrug (e.g., gancyclovir). A herpes simplex virus thymidine kinase (HSVtk) gene is an example of a cytotoxic gene.

Any type of mammal having a cancer can be treated using the methods and materials provided herein including, without limitation, mice, rats, dogs, cats, horses, cows, pigs, monkeys, and humans. Any appropriate method can be used to administer a composition provided herein to a mammal. For example, a composition provided herein can be administered via injection (e.g., intramuscular injection, intradermal injection, or intravenous injection).

A composition comprising a nucleic acid encoding a cytotoxic polypeptide, a nucleic acid encoding a chaperone polypeptide, and nucleic acid encoding CD40L polypeptide can be administered following surgical resection of a tumor. In some cases, a composition provided herein can be administered prior to surgical resection of a tumor.

Before administering the composition described herein to a mammal, the mammal can be assessed to determine whether or not the mammal has a cancer. Any suitable method can be used to determine whether or not a mammal has cancer. For example, a mammal (e.g., a human) can be identified as having a cancer using standard diagnostic techniques. In some cases, a tissue biopsy can be collected and analyzed to determine whether or not a mammal has a cancer.

After identifying a mammal as having a cancer, the mammal can be treated with the composition described herein. Such compositions can be administered to a mammal in any amount, at any frequency, and for any duration effective to achieve a desired outcome (e.g., to reduce the progression rate of melanoma or to induce prostate cancer regression). In some cases, the composition described herein can be administered to a mammal to reduce the progression rate of a cancer by 5, 10, 25, 50, 75, or 100 percent. For example, the progression rate can be reduced such that no additional cancer progression is detected. Any standard method can be used to determine whether or not the progression rate of a cancer is reduced. For example, the progression rate of a cancer can be assessed by measuring a tumor at different time points and determining the size of the tumor. The size of the tumor determined at different times can be compared to determine the progression rate. After treatment with a composition provided herein, the progression rate can be determined again over another time interval. In some cases, the stage of a cancer after treatment can be determined and compared to the stage before treatment to determine whether or not the progression rate is reduced.

An effective amount of a composition provided herein can be any amount that reduces the progression rate of a cancer without producing significant toxicity to the mammal. Typically, an effective amount can be any amount greater than or equal to about 10 μg each of a nucleic acid molecule encoding a cytotoxic polypeptide (e.g., polypeptide encoded by a transcriptionally targeted cytotoxic gene), a nucleic acid molecule encoding a chaperone polypeptide, and a nucleic acid molecule encoding CD40L polypeptide provided that that amount does not induce significant toxicity to the mammal upon administration. In some cases, the effective amount can be between 50 μg and 500 μg. In some cases, a composition can be administered such that the mammal receives between 50 ng and 1 g of a nucleic acid molecule encoding a cytotoxic polypeptide, a nucleic acid molecule encoding a chaperone polypeptide, and a nucleic acid molecule encoding CD40L polypeptide each. If a particular mammal fails to respond to a particular amount, then the amount can be increased by, for example, ten fold. After receiving this higher concentration, the mammal can be monitored for both responsiveness to the treatment and toxicity symptoms, and adjustments made accordingly. When injected, an effective amount can be between 50 μg and 100 μg. The effective amount can remain constant or can be adjusted as a sliding scale or variable dose depending on the mammal's response to treatment.

Various factors can influence the actual effective amount used for a particular application. For example, the frequency of administration, duration of treatment, use of multiple treatment agents, route of administration, and severity of the cancer may require an increase or decrease in the actual effective amount administered.

The frequency of administration can be any frequency that reduces the progression rate of a cancer without producing significant toxicity to the mammal. For example, the frequency of administration can be from about four times a day to about once every other month, or from about once a day to about once a month, or from about one every other day to about once a week. In addition, the frequency of administration can remain constant or can be variable during the duration of treatment. Any of the compositions provided herein can be administered daily, twice a day, five days a week, or three days a week. Such compositions can be administered for five days, 10 days, three weeks, four weeks, eight weeks, 48 weeks, one year, 18 months, two years, three years, or five years. A course of treatment with the disclosed compositions can include rest periods. For example, a composition comprising a nucleic acid molecule encoding cytotoxic polypeptide, a nucleic acid molecule encoding a chaperone polypeptide, and a nucleic acid molecule encoding CD40L polypeptide can be administered for five days followed by a nine-day rest period, and such a regimen can be repeated multiple times. As with the effective amount, various factors can influence the actual frequency of administration used for a particular application. For example, the effective amount, duration of treatment, use of multiple treatment agents, route of administration, and severity of the cancer may require an increase or decrease in administration frequency.

An effective duration for administering a composition provided herein can be any duration that reduces the progression rate of cancer without producing significant toxicity to the mammal. Thus, the effective duration can vary from several days to several weeks, months, or years. In general, the effective duration for the treatment of a cancer can range in duration from several days to several months. In some cases, an effective duration can be for as long as an individual mammal is alive. Multiple factors can influence the actual effective duration used for a particular treatment. For example, an effective duration can vary with the frequency of administration, effective amount, use of multiple treatment agents, route of administration, and severity of the cancer.

After administering a composition provided herein to a mammal, the mammal can be monitored to determine whether or not the cancer was treated. For example, a mammal can be assessed after treatment to determine whether or not the progression rate of melanoma was reduced or stopped). As described herein, any method can be used to assess progression rates.

The invention will be further described in the following examples, which do not limit the scope of the invention described in the claims.

### EXAMPLES

### Example 1

### Induction of Autoimmunity to Treat Cancer

### Cell Lines, Plasmids and Viruses

The murine melanoma B16.F1 tumor cell line used in the following experiments was described elsewhere (Linardakis et al., Cancer Res., 62:5495-5504 (2002)). The plasmids used in these experiments were described elsewhere (Daniels et al., Nature Biotechnol., 22:1125-1132 (2004)). Briefly, the Tyr-HSVtk plasmid contains a hybrid promoter of three tandem copies of a 200 by element of the murine tyrosinase enhancer (Ganss et al., Embo. J., 13:3083-3093 (1994)) upstream of a 270 by fragment of the tyrosinase promoter (Vile and Hart, Cancer Res., 53:962-967 (1993)) to drive expression of the HSVtk gene (Vile and Hart, Cancer Res., 53:3860-3864 (1993)). In CMV-hsp70, the murine hsp70 gene (Melcher et al., Nature Medicine, 4:581-587 (1998)) is driven by the CMV promoter in pCR3.1 (Invitrogen). In pCD40L, the murine CD40L gene is driven by the CMV promoter. The adenovirus expressing murine TNF-α was obtained from Dr Zhou Xing, McMaster University, Canada.

### Reverse Transcriptase Polymerase Chain Reaction

Skin samples at the site of plasmid injection were snap frozen in liquid nitrogen. RNA was prepared with the QIAGEN RNA extraction kit. One mg total cellular RNA was reverse transcribed in a 20 μL volume using oligo-(dT) as a primer. A cDNA equivalent of 1 ng RNA was amplified by PCR for a variety of murine cytokines or melanoma/melanocyte antigens as described elsewhere (Linardakis et al., supra; and Vile et al., Int. J. Cancer, 71:267-274 (1997)).

### Splenocyte Preparation

Splenocytes enriched in lymphocytes were prepared from spleens by standard techniques (Coligan et al., 1998, Current Protocols in Immunology. Wiley and Sons, Inc.) and Lympholyte-M density separation (Cedarlane, Ontario, Calif.). CD8+ T cells were purified from spleens using the MACS CD8a (Ly-2) Microbead magnetic cell sorting system (Miltenyi Biotec, Auburn, Calif.).

### Antigen Presentation Assays and Tetramers

Freshly purified splenocyte populations were washed in PBS and pulsed with 5 μM peptide for 2 hours at 37° C. before being incubated with purified CD8+ T cells or splenocytes harvested from mice from the appropriate treatment groups. 72 hours later splenocytes were subjected to FACS analysis or cell free supernatants were tested for IFN-γ by ELISA (Pharmingen). The synthetic, H-2 Kb-restricted polypeptides hgp100₂₅₋₃₃, KVPRNQDWL (SEQ ID NO:1), TRP-2₁₈₀₋₁₈₈ SVYDFFVWL (SEQ ID NO:2) (Dyall et al., J. Exp. Med., 188:1553-1561 (1998)) and Ova SIINFEKL (SEQ ID NO:3) (Hogquist et al., Cell, 76:17 (1994)) were synthesized at the Mayo Foundation Core facility. An altered ligand from hgp100 was used, as opposed to the murine epitope, because it has been shown to be presented more effectively in the context of H-2 Kb⁻ restricted murine DC (Overwijk et al., J. Exp. Med., 198:569-580 (2003)). Tetramers bound with the H-2 Kb-restricted SIINFEKL (SEQ ID NO:3) or TRP-2₁₈₀₋₁₈₈ SVYDFFVWL (SEQ ID NO:2) polypeptides were commercially available from Beckman Coulter, Chino, Calif.

### ELISPOT Analysis for IFN-γ Secretion

IFN-γ ELISPOT assays were purchased from Pharmingen and used as recommended. Splenocytes were stimulated in the presence of the appropriate polypeptide in triplicate cultures at a density of 250,000 splenocytes per well. Spot numbers were determined 72 hours later by computer assisted image analyzer.

### In Vivo Studies

C57BL/10ScNJ mice contain a deletion of the Tlr4 gene. B6; 12956-Tnftm1Gkl/J mice are TNF-deficient. C57BL/10ScNJ and B6; 12956-Tnftm1Gkl/J mice were purchased from the Jackson Laboratory (stock numbers 003752 and 003008, respectively). C57BL/6 mice were age and sex-matched for individual experiments. To establish subcutaneous tumors, 2×10⁵ B16 cells were injected s.c. (100 μL) into the flank region. Animals were examined daily until the tumor became palpable, whereafter the diameter, in two dimensions, was measured thrice weekly using calipers. Animals were killed when tumor size was approximately 1.0×1.0 cm in two perpendicular directions. In all experiments, 10 mice per group were used unless indicated otherwise in the figures.

Plasmid injections were carried out by intradermal injection (Daniels et al., supra; and Bonnotte et al., Cancer Res., 63:2145-2149 (2003)) in a final volume of 50 μL in PBS. For LN/cell tracking studies, Cell Tracker Green (5′-chloro-methyl-fluorescein diacetate) (Molecular Probes, Eugene, Oreg.) was added to the plasmid mix at a final concentration of 25 μM prior to intradermal injections.

### Tumor Treatment Protocols

For protocols aimed at treating established subcutaneous tumors, 2×10⁵ B16 cells were seeded subcutaneously in the right flank of C57BL/6 mice (day 0). At the appropriate day following tumor seeding, 20 μg or 30 μg of plasmid DNA was injected intradermally on the contralateral flank. GCV at 50 mg/kg was administered i.p. A 3-day established tumor was usually palpable under the skin, and a 9 day established tumor was usually about 0.3-0.4 cm in its longest diameter.

### Statistics

Data from the animal studies were analyzed by the logrank test (Altman, D. G. 1991. Analysis of survival times. In: Practical Statistics for Medical Research, pp. 365-395). Statistical significance was determined at the level of p<0.05.

### Hsp70 Induces Anti-Tumor Immunity Through TLR-4-Dependent Signaling

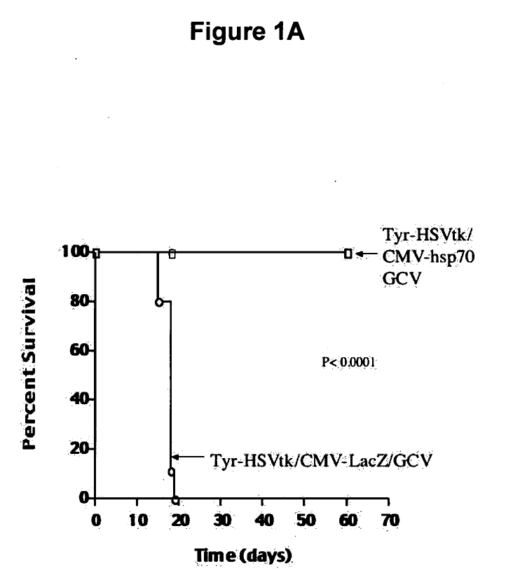

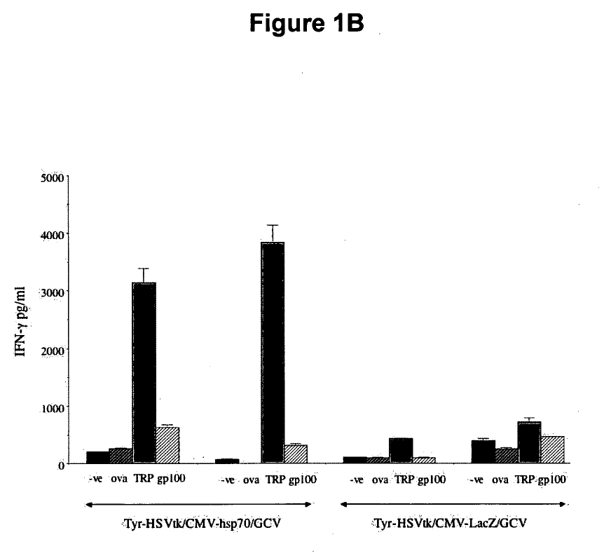

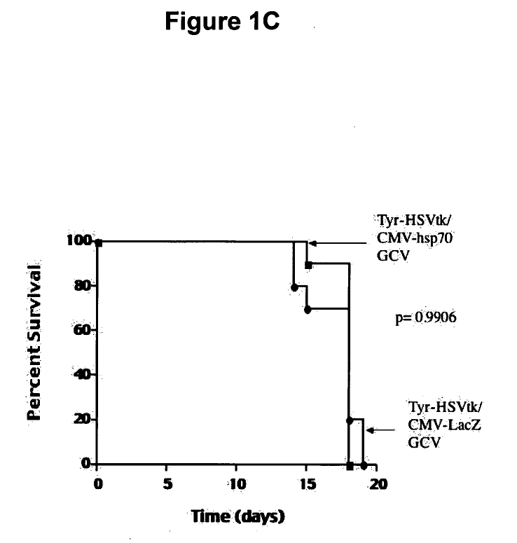

Three rounds of Tyr-HSVtk/CMV-hsp70/GCV treatment (a total of 9 intradermal plasmid injections and 15 i.p. injections of GCV) cures 70-100% of mice bearing three day established subcutaneous B16 tumors on the contralateral flank (Daniels et al., supra; and Sanchez-Perez et al., Cancer Res., 65:2009-2017 (2005)) (FIG. 1A). Consistent with anti-tumor therapy, i.d. injection of the hsp70 plasmid was used for priming TRP-2 specific responses in the spleens of vaccinated mice (FIG. 1B), confirming that TRP-2 and tyrosinase, but not gp100, are targets of the curative T cell responses raised in vivo by inflammatory killing of melanocytes.

Hsp70 was reported to act as a chaperone of immunogenic polypeptides, a cytokine, an immunogen, a maturation agent for dendritic cells, and as an inducer of pro-inflammatory cytokines from monocytes following ligation to Toll Like Receptors (TLR) 2 and 4. To understand which of these possible activities hsp70 is exerting, the protocol of inflammatory melanocyte killing in mice lacking key elements of these effector responses was tested. Whereas C57Bl/6 mice bearing 3 day established B16 tumors were cured by intradermal injections of Tyr-HSVtk/CMV-hsp70/GCV (FIG. 1A), C57BL/10ScNJ mice, which carry a deletion of the Tlr4 gene, were completely unable to reject their tumors and died as rapidly as control treated C57BL/6 mice (FIG. 1C). These results suggest that hsp70 plays a major role in the in vivo therapy by signaling through TLR-4 on the surface of endogenous cells.

Local Expression of Hsp70 Induces Priming of Anti-Melanoma/Melanocyte Responses through induction of TNF-α

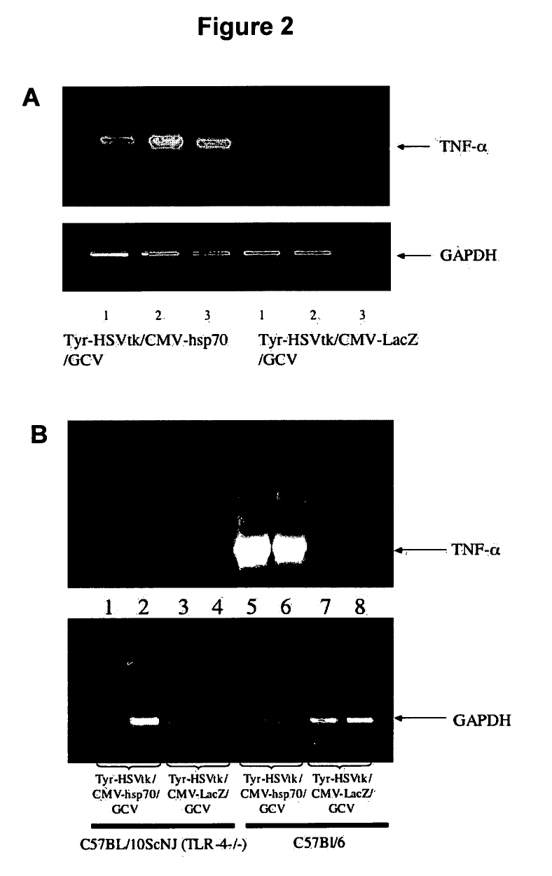

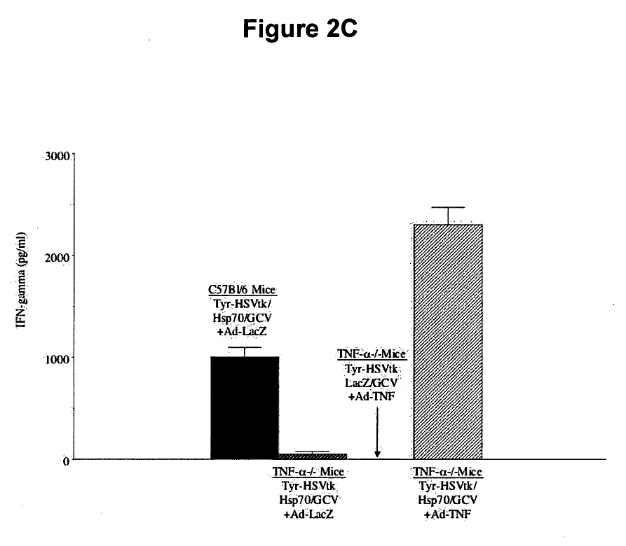

The site of hsp70 injection was examined for the expression of cytokines. Of the 7 different cytokines tested, TNF-α correlated consistently with the expression of hsp70 (FIG. 2A). In contrast, hsp70 expression at the site of melanocyte killing was unable to induce local TNF-α expression in C57BL/10ScNJ mice lacking TLR-4 signaling (FIG. 2B). Thus, hsp70 expression appears to induce local immune activation through TNF-α induction as an element to the in vivo, CD8+ T cell mediated therapy of B16 tumors. In this respect, it was observed that, whereas the Tyr-HSVtk/CMV-hsp70/GCV treatment effectively primed TRP-2 specific responses in C57BL/6 mice (FIGS. 1B and 2C), these effects were lost in B6; 129S6-Tnftm1Gkl/J TNF-α−/− mice (FIG. 2C). The local provision of TNF-α by delivery of an adenovirus expressing TNF-α at the site of plasmid injection was able to rescue the ability of these mice to generate TRP-2 specific responses but only if hsp70 was provided in the plasmid injections (FIG. 2C).

### Hsp70 Expression Induces Trafficking of an APC-Like Population to the Draining Lymph Node

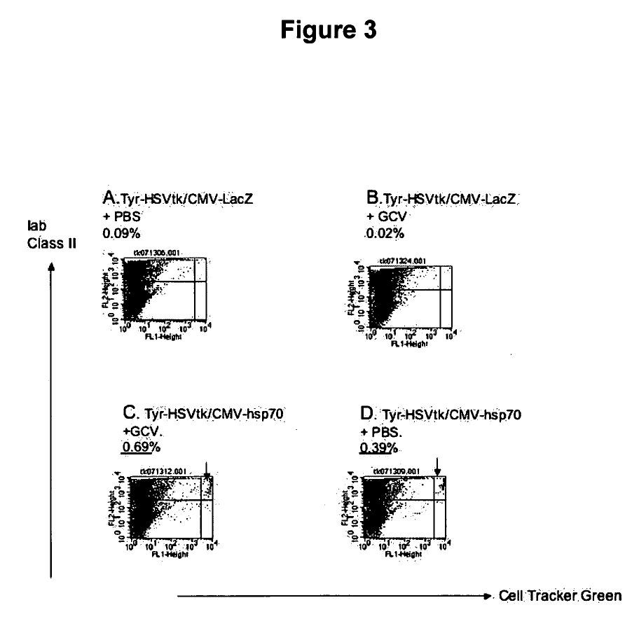

Local immune activation by hsp70, through TLR-4 mediated signaling and TNF-α induction, appears to induce migration of APC from the site of plasmid injection to the lymph nodes (LN). To test this, Cell Tracker Green (CTG) dye-labeled cells were tracked from the site of injection to the draining LN. No CTG+ve cells were detected in draining LN following i.d. injection of CTG alone, or with any plasmid combination in which hsp70 was not present (FIGS. 3A and B). When hsp70 was expressed locally, MHC Class II (Hi), CTG+ve cells trafficked to the LN, irrespective of whether GCV was administered as well (arrows, FIGS. 3 C and D), consistent with this being a population of activated APC. This CTG+/MHC Class II (Hi) population was further characterized and shown to consist of between 55-60% MAC3+ve cells (FIG. 3F) and ˜40% Mac3-ve, CD11c+ve cells.

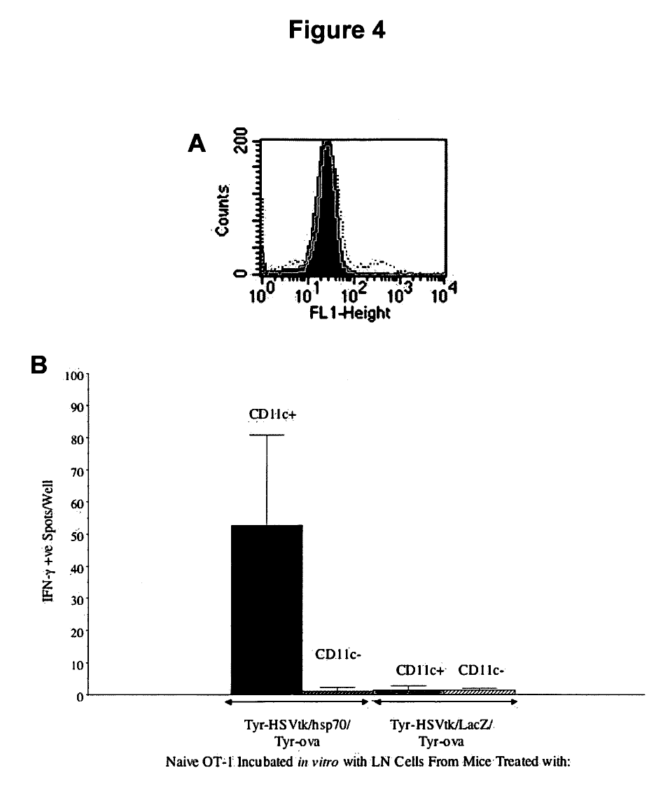

To test the functional relevance of this LN migration, a plasmid (Tyr-ova) was co-delivered in which the cDNA of the model chick ovalbumin antigen, expressed from the tyrosinase promoter, is only expressed in melanocytes. CD8+ T cells specific for the H-2 Kb-restricted SIINFEKL (SEQ ID NO:3) polypeptide of ova could be detected in LN by tetramer analysis, but only if pTyr-ova was co-injected with Tyr-HSVtk+GCV (to kill melanocytes and release ova antigen) and CMV-hsp70 (consistent with migration to the LN of a putative APC population) (FIG. 4A). Priming of naïve T cell responses to the TRP-2 antigen in these assays were not detected.

Because the Mac3 marker is not truly specific for macrophages, transgenic OT-1 T cells (specific for H-2K^{b}-restricted SIINFEKL (SEQ ID NO:3)) were used to monitor which of the Mac3+ve, or CD11c+ve, cell populations migrating to the LN are presenting the melanocyte-derived (ova) antigen. FIG. 4B shows that the SIINFEKL (SEQ ID NO:3) epitope of the ova antigen, expressed from the melanocyte specific tyrosinase promoter, was presented almost exclusively by the CD11c+ve population of cells which hsp70 induces to migrate to the draining lymph node.

### Hsp70-Induced LN Trafficking is Critical to Therapeutic Efficacy.

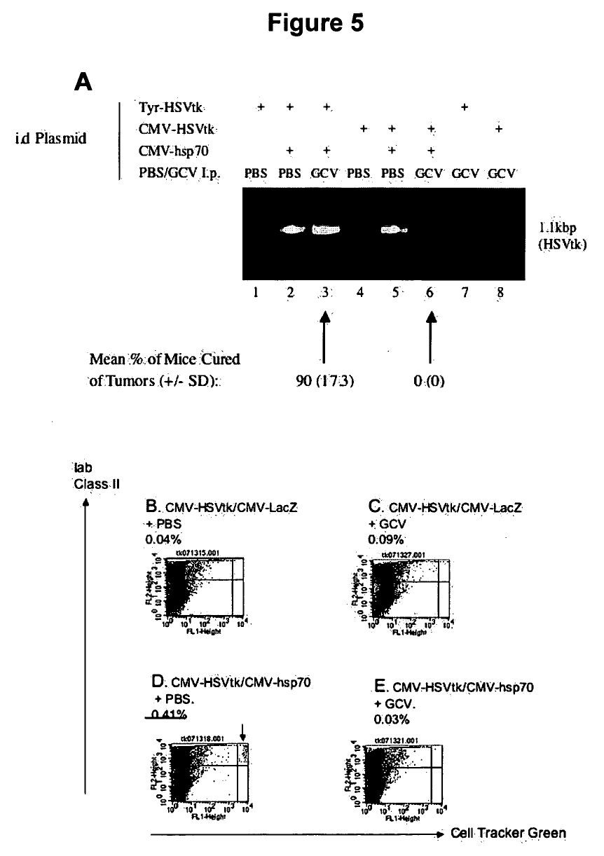

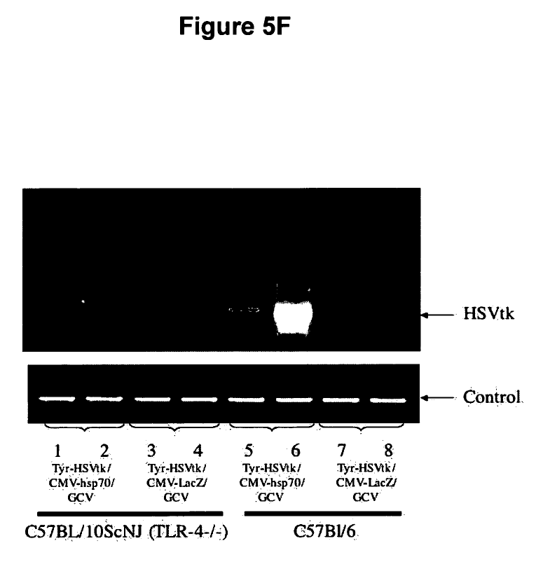

When the Tyr-HSVtk plasmid was replaced with a CMV-HSVtk plasmid (HSVtk cDNA expressed by the CMV promoter) in the therapeutic protocol of FIG. 1A, a complete loss of therapy of established B16 tumors was consistently observed (FIG. 5A), even though levels of expression of HSVtk were directly comparable between the two plasmids in melanocyte-derived cell types. PCR from genomic DNA of LN cells following i.d. plasmid injections revealed that the injected HSVtk gene could be detected in draining LN cells (FIG. 5A), but only in mice in which hsp70 had been present (FIG. 5A). These results were consistent with cell tracking studies presented in FIG. 3 showing the involvement of hsp70 in promoting LN migration of APC dependent upon TLR-4-mediated, TNF-α mechanisms. Consistent with the observation of loss of therapy when the Tyr-HSVtk plasmid was replaced with the CMV-HSVtk plasmid, no PCR signal for the HSVtk transgene could be detected in LN of mice injected with CMV-HSVtk/CMV-hsp70 and given GCV treatment (FIG. 5A). Similarly, CTG-labeled, Class II Hi cells were detected in the LN following i.d. plasmid injections with the CMV-HSVtk plasmid only if hsp70 were co-expressed (FIG. 5B-D) and if PBS was administered (FIG. 5D). This LN migrating population was lost, even if hsp70 was present, if GCV was used instead of PBS (FIG. 5E). The HSVtk transgene was also not detected by PCR in the LN following i.d. Tyr-HSVtk/CMV-hsp70/GCV injections into C57BL/10ScNJ mice, which carry a deletion of the Tlr4 gene (FIG. 5F), confirming the involvement of hsp70/TLR-4 signaling in vivo to promote the migration of cells carrying melanocyte derived antigen to the LN. Overall, these data are consistent with the hypothesis that CMV-HSVtk/GCV kills hsp70-activated APC carrying melanocyte antigens from the site of melanocyte destruction to the LN, and explain why Tyr-HSVtk/hsp70/GCV, but not CMV-HSVtk/hsp70/GCV, induces tumor regressions.

### Inflammatory Killing of Melanocytes is Enhanced by Additional T Cell Costimulation.

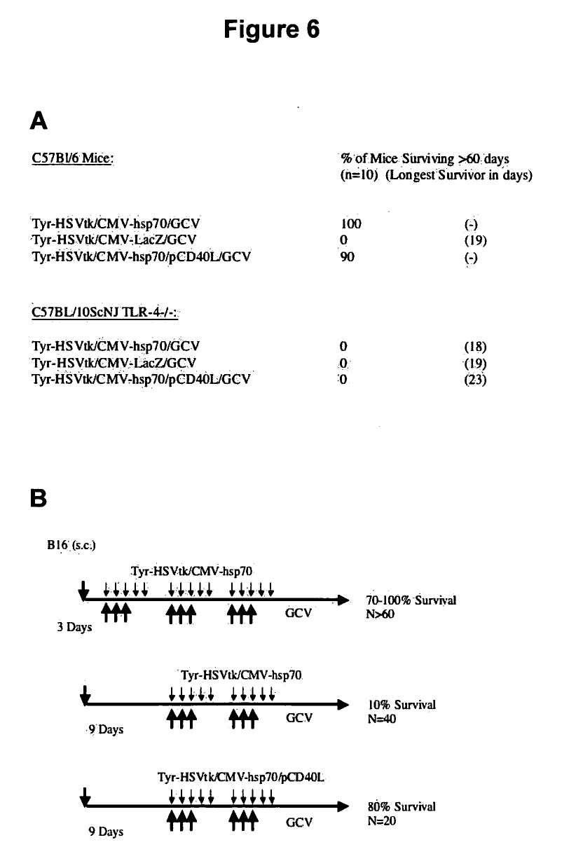

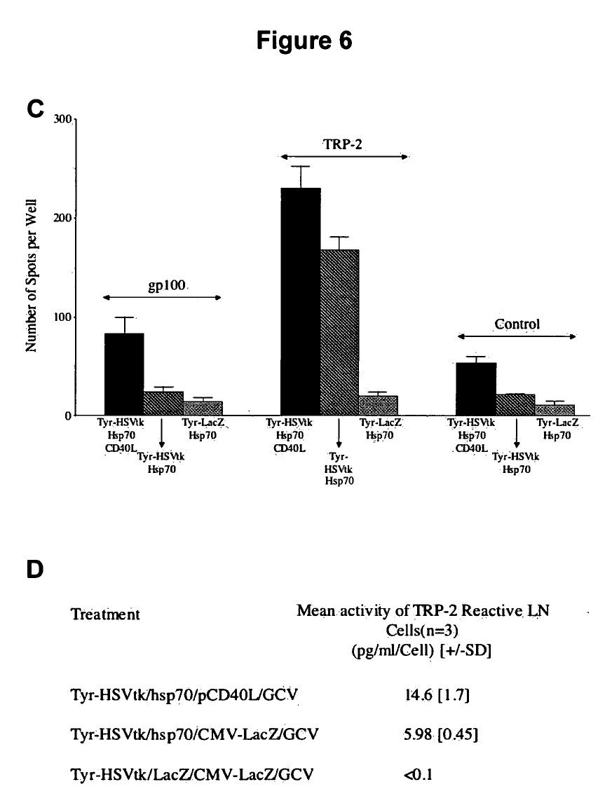

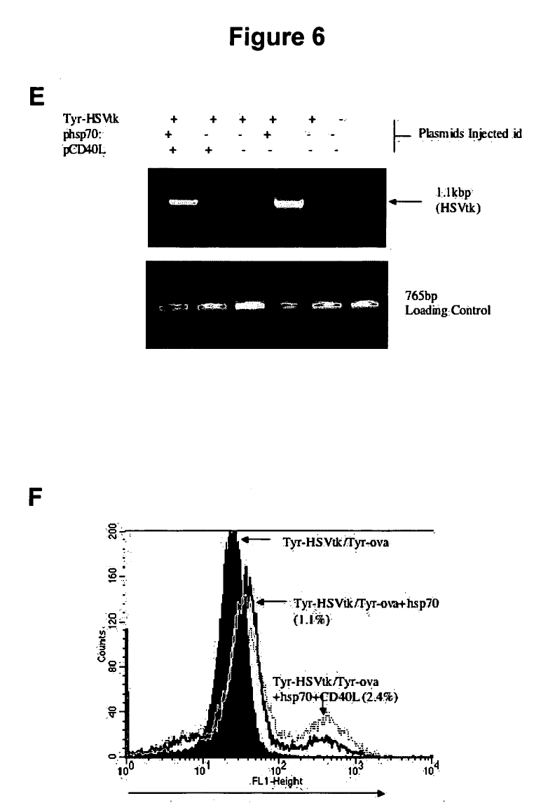

It was investigated whether addition of CD40 ligation could replace hsp70, or whether it would enhance the quality, and/or quantity, of the T cell response against melanocyte antigens. A plasmid expressing CD40L from the CMV promoter (pCD40L) was added to the regimen of plasmid injections. When pCD40L was added to the curative protocol of FIG. 1A, there was no significant inhibition of treatment efficacy compared to using hsp70 alone (FIG. 6A). Moreover, the presence of pCD40L in the plasmid treatments gave no significant added survival benefit to C57BL/10ScNJ TLR-4−/− mice bearing 3 day established B16 tumors compared to treatment with Tyr-HSVtk/CMV-hsp70/GCV alone (FIG. 6A). Taken together, these data indicate that pCD40L could neither inhibit, nor replace, the activity of hsp70 in either wild-type or TLR4−/− mice.

Whether addition of pCD40L could enhance the activity of hsp70-mediated inflammatory melanocyte killing was tested. Animals bearing 9 day established s.c. B16 tumors treated with only two rounds of Tyr-HSVtk/CMV-hsp70 and 10 i.p. injections of GCV, typically survive longer than controlled treated animals but nearly all eventually succumb to disease (Daniels et al., supra; and Sanchez-Perez et al., supra; FIG. 6B). In contrast, over several different experiments, 80% of animals treated with Tyr-HSVtk/CMV-hsp70/pCD40L (10 μg of each plasmid) rejected their tumors and survived>60 days following tumor seeding (FIG. 6B). pCD40L was unable to substitute for hsp70. Thus, whereas mice treated with 9 injections of Tyr-HSVtk/CMV-hsp70 plasmid were cured of 3 day tumors (FIG. 1), 0/20 mice treated with Tyr-HSVtk/pCD40L were cured and tumors grew as rapidly as in control treated animals.

Co-Expression of pCD40L Increases the Number and Potency of TRP-2 Specific T Cells.

Hsp70-mediated inflammatory killing of melanocytes primes T cell responses specific to the TRP-2, but not gp100, antigens (Daniels et al., supra; and Sanchez-Perez et al., supra). Consistent with the increased therapeutic potential of expression of CD40L at the injection site, ELISPOT data indicated that there was a modest, but consistently significant (p<0.01), increase in the frequency of TRP-2 specific splenocytes generated in vivo 8 days following the first of three injections of Tyr-HSVtk/CMV-hsp70/pCD40L+GCV compared to treatment with Tyr-HSVtk/CMV-hsp70+GCV (FIG. 6C). Combined with IFN-γ ELISA analysis, close to a three fold increase was observed in the specific activity of the TRP-2 specific splenocytes generated following i.d. injection of Tyr-HSVtk/CMV-hsp70/pCD40L+GCV compared to Tyr-HSVtk/CMV-hsp70+GCV (FIG. 6D). Inclusion of CD40L in the plasmid regimen also enhanced epitope spreading (Ribas et al., Trends Immunol., 24:58-61 (2003)) in that splenocytes specific for gp100 could now be detected in Tyr-HSVtk/CMV-hsp70/pCD40L/GCV treated mice (FIG. 6C) whereas gp100 reactive T cells were not detectable in Tyr-HSVtk/CMV-hsp70/GCV-treated mice (FIG. 6C; Daniels et al., supra; and Sanchez-Perez et al., supra).

Injection of pCD40L did not increase the number of cell tracker green cells, or CD11c+ve cells, detected in the LN using either the PCR detection method (FIG. 6E) or by flow cytometry in the cell migration assay described in FIG. 2. Inclusion of the pTyr-ova plasmid into the plasmid injection regimen significantly increased the number of SIINFEKL- (SEQ ID NO:3) specific T cells detected in the LN compared to when pCD40L was absent (p<0.001) (FIG. 6F), confirming again that ligation of CD40 on putative APC increases the number of antigen specific T cells primed in the draining lymph node.

### CD40L Enhances Anti-Melanocyte Responses and Immunological Memory In Vivo.

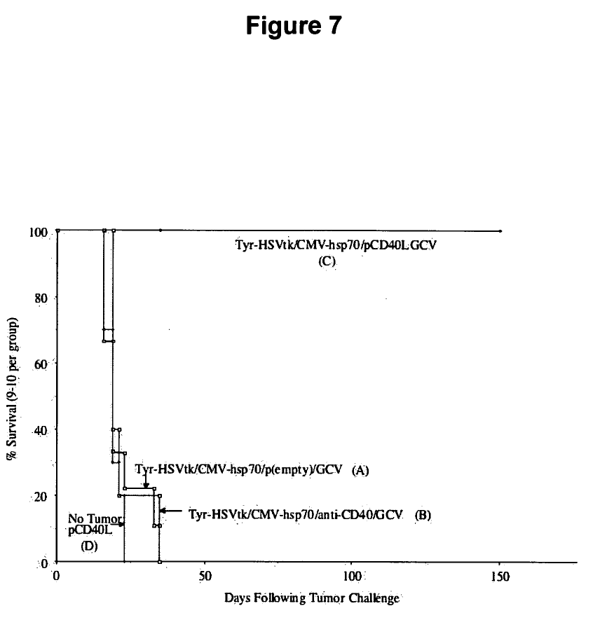

In animals cured of 3 day established tumors by 9 injections of Tyr-HSVtk/CMV-hsp70/GCV, development of autoimmune disease was difficult to detect; only mice depleted of CD25⁺ T cells, which received Tyr-HSVtk/CMV-hsp70/GCV treatment, but never saw tumors, developed localized areas of depigmentation. In addition, long-term survivors (>100 days) could not reject re-challenge with B16. This indicates that the CD8⁺ T cell response from Tyr-HSVtk/CMV-hsp70/GCV therapy is short lived, due at least in part to the induction of putative suppressor cells in the CD4+CD25+ compartment (Daniels et al., supra). In contrast, animals cured by Tyr-HSVtk/CMV-hsp70/pCD40L intradermal injections developed alopecia-like symptoms with often severe but patchy hair loss across their abdomens. In addition, these mice were often unable to re-grow their hair in the shaved areas where the initial injections had been performed. Moreover, mice surviving the 9 day established tumors following Tyr-HSVtk/CMV-hsp70/pCD40L/GCV treatment developed stringent memory in 100% of the survivors (FIG. 7), and none of the cured mice has developed recurrent tumor growth up to 9 months following tumor challenge. Systemic administration of an anti-CD40 antibody (FGK45 at 50 μg i.p.) was ineffective (Group B, FIG. 7). These data indicate that pCD40L enhances the development of autoimmune disease that was observed following Tyr-HSVtk/CMV-hsp70/GCV therapy alone, and which was demonstrated to be controlled in part by both the presence of tumor and the activity of regulatory T cells.

### Example 2

### Induction of hsp70-Mediated Th17 Autoimmunity as Immunotherapy for Metastatic Prostate Cancer

### Cell Lines, Plasmids, and Viruses

Transgenic adenocarcinoma of the mouse prostate (TRAMP)-C2 (TC2) cells were derived from a prostate tumor that arose in a TRAMP mouse. These cell lines express a variety of prostate-specific genes, including PSMA, Hoxb-13, and NKX3.1. TC2 cells grow in an androgen-independent manner and have a reduced level of expression of MHC class I, which can be up-regulated by IFN-γ, making them susceptible to specific lysis by CTL. TC2 tumors are routinely grown in C57Bl/6 male mice. The murine melanoma B16.F1 tumor cell line has been previously described (supra). Cell lines were grown in DMEM (Invitrogen/Life Technologies, Carlsbad, Calif.) supplemented with 10% (v/v) FCS (Invitrogen/Life Technologies) and L-glutamine (Invitrogen/Life Technologies). All cell lines were monitored routinely and found to be free of Mycoplasma infection.

The replication-defective adenoviral vectors used in this study were all E1 deleted serotype 5 vectors that contains the cytomegalovirus (CMV) immediate-early gene promoter-enhancer driving the inserted transgene. Ad-VSV-G expresses the cDNA of the fusogenic membrane G glycoprotein of vesicular stomatitis virus (VSV-G; Linardakis et al., supra; Bateman et al., Cancer Res., 62:5466-6578 (2002); and Higuchi et al., Cancer Res. 60:6396-6402 (2000)); Ad-hsp70 contains the cDNA of the inducible murine heat shock protein 70 gene (Melcher et al., Hum. Gene Ther., 10:1431-1442 (1999)); and Ad-GFP contains the cDNA of the green fluorescent protein gene (Ahmed et al., Nat. Biotechnol., 21:771-777 (2003)). In the CMV-ova plasmid (CMV-ova), the ovalbumin gene is driven by the CMV promoter in pCR3.1 (Invitrogen).

### Histopathology of Tumor Sections

Prostates were harvested and fixed in 10% Formalin in PBS, then paraffin embedded and sectioned. Hematoxylin and eosin- (H&E-) stained sections were prepared for analysis of tissue destruction and gross infiltrate. Two independent pathologists examined H&E sections, blinded to the experimental design, and scored the degree of necrosis.

### Reverse Transcriptase PCR

Organ samples were snap frozen in liquid nitrogen. RNA was prepared using a Qiagen (Valencia, Calif.) RNA extraction kit. One microgram of total cellular RNA was reverse transcribed in a 20 μL volume using oligo-(dT) as a primer. A cDNA equivalent of 1 ng RNA was amplified by PCR for a variety of murine cytokines or vector-derived transgenes as described previously (Linardakis et al., supra; and Vile et al., supra).

Treg-Mediated Inhibition of IFNγ Secretion from Activated T Cells

OT-I mice are transgenic mice whose T cells express the V_{α2} chain of the transgenic OT-I T-cell receptor that specifically recognizes the SIINFEKL (SEQ ID NO:3) peptide from the chicken ovalbumin protein (ova) in the context of H-2K^{b} as expressed by B16ova tumor cells (Hogquist et al., supra). For preparation of naive OT-I T cells, spleen and lymph nodes from OT-I-transgenic mice were combined and crushed through a 100-nm filter to prepare a single-cell suspension. RBC were removed by a 2-minute incubation in ACK buffer (sterile dH₂O containing 0.15 mol/L NH₄Cl, 1.0 mmol/L KHCO₃, and 0.1 mmol/L EDTA adjusted to pH 7.2-7.4). OT-I T cells were activated by incubation of splenocyte populations with the cognate antigen recognized by the OT-I T cells. Single-cell suspensions from spleen and lymph nodes were adjusted to 1.0×10⁶ cells/mL in Iscove's modified Dulbecco's medium plus 5% FCS, 10⁵ mol/L 2-ME, 100 units/mL penicillin, and 100 μg/mL streptomycin and stimulated with 1 μg/mL SIINFEKL (SEQ ID NO:3) peptide and 50 IU/mL human IL-2 (Mayo Clinic Pharmacy). This routinely induces large amounts of IFN-γ to be expressed from the activated OT-I T cells.

To assay for the presence of T-cell suppressive (Treg) activity within splenocyte populations from intraprostatically injected mice, 250,000 freshly harvested splenocytes from treatment groups were plated along with 10⁵ naive OT-1 CD8⁺ T cells in the presence of either no added peptide, an irrelevant nonactivating peptide (TRP-2₁₈₀₁₈₈ SVYDFFVWL (SEQ ID NO:2; Dyall et al., supra), or with the synthetic H-2K^{b}-restricted ova peptide SIINFEKL (SEQ ID NO:3; Hogquist et al., supra) in tissue culture wells. Splenocyte/OT-I cocultures were stimulated in triplicate and supernatants were assayed for IFN-γ production by ELISA. The degree of suppressive activity in the test splenocyte cultures was reflected by their ability to inhibit the IFN-γ response of the naïve OT-I T cells when presented with their cognate, activating SIINFEKL (SEQ ID NO:3) antigen. The dependence of any such T-cell suppressive activity on expression of transforming growth factor-β (TGF-β; Thomas and Massague, Cancer Cell, 8:369-380 (2005)) was assayed using the recombinant human TGF-β sRII/Fc chimera (R&D Systems, Minneapolis Minn.), a 159 amino acid extracellular domain of human TGF-β receptor type II fused to the Fc region of human IgG 1.

### Antigen Priming Assays—Splenocyte Preparation and Antigen Presentation

Splenocytes enriched in lymphocytes were prepared from spleens from treated/vaccinated animals by standard techniques (Coligan et al., Current Protocols in Immunology, Wiley and Sons, Inc. (1998)). Freshly purified splenocyte populations were washed in PBS and either incubated with target tumor cells (TC2 or B16) typically at ratios of 100:1, 10:1, or 1:1 or, where appropriate, were pulsed with 1 μg/mL of the target peptide for which antigen specificity of response was being tested [SIINFEKL (SEQ ID NO:3) for induced responses to ova (Hogquist et al., supra) or TRP-2₁₈₀₁₈₈ SVYDFFVWL (SEQ ID NO:2; Dyall et al., supra) as the negative irrelevant antigen control]. Forty-eight to seventy-two hours later, cell-free supernatants were tested for IFN-γ by ELISA (PharMingen). The synthetic H-2 Kb-restricted peptides TRP-2₁₈₀₁₈₈ SVYDFFVWL (SEQ ID NO:2) and Ova SIINFEKL (SEQ ID NO:3) were synthesized at the Mayo Foundation Core facility.

### ELISA Analysis for IFNγ Secretion

For ELISA, cell-free supernatants were collected from sample wells and tested by specific ELISA for IFN-γ or IL-6 (BD OptEIA IFN-γ; BD Biosciences, San Jose, Calif.) or IL-17 (R&D Systems) according to the manufacturers' instructions.

### In Vivo Studies

C57Bl/6 mice or B6.129S2-IL6^{tm1Kopf}/J [IL-6 knockout (IL-6KO); Jackson; No. 002650] were purchased from The Jackson Laboratory (Bar Harbor, Me.) at ages 6 to 8 weeks. To establish s.c. tumors, 2×10⁵ B16 cells or 2×10⁶ TC2 cells in 100 μL of PBS were injected into the flank of mice. Intraprostatic injections (50 μL) were performed on mice under anesthetic, typically at day 6 after tumor establishment. For survival studies, tumor diameter in two dimensions was measured thrice weekly using calipers, and mice were killed when tumor size was about 1.0×1.0 cm in two perpendicular directions.

Immune cell depletions were performed by i.p. injections (0.1 mg per mouse) of anti-CD8 (Lyt 2.43) and anti-CD4 (GK1.5), both from the Monoclonal Antibody Core Facility, Mayo Clinic; and IgG control (ChromPure Rat IgG; Jackson ImmunoResearch, West Grove, Pa.) at day 4 after tumor implantation and then weekly thereafter. For Treg depletion, 0.5 mg of PC-61 antibody (Monoclonal Antibody Core Facility, Mayo Clinic) per mouse was given i.p. 4 days after tumor implantation and 2 days before the first viral injection. Fluorescence-activated cell sorting analysis of spleens and lymph nodes confirmed subset specific depletions.

### Statistics

Survival data from the animal studies was analyzed using the log-rank test (Altman, supra), and the two-sample unequal variance Student's t test analysis was applied for in vitro assays. Statistical significance was determined at the level of P value of <0.05.

### Inflammatory Killing of Normal Prostate Induces Ongoing Autoimmune Destruction

Previously, plasmids expressing the HSVtk suicide gene and hsp70 were used to target killing of normal melanocytes in the skin (Sanchez-Perez et al., J. Immunol. 177:4168-4177 (2006); Daniels et al., supra; and Sanchez-Perez et al. (2005), supra). Because HSVtk requires active division of target cells for cytotoxicity, an adenoviral vector expressing VSV-G, the fusogenic membrane glycoprotein (FMG) from VSV (Linardakis et al., supra), was used in the current studies to induce killing of normal prostate cells. It has been shown that killing induced by fusion of cells using viral FMG can be potently immunogenic through the fusion of cells into multinucleated syncytia (Linardakis et al., supra; and Bateman et al., supra). In addition, a second adenoviral vector was used to express the murine hsp70 gene (Melcher et al., supra).

Direct intraprostatic injection of an adenovirus-expressing GFP (Ad-GFP) did not induce any detectable lasting damage to the prostates of C57Bl/6 mice either in terms of the architecture of the organ or immune infiltration. Injection of Ad-hsp70 alone induced an inflammatory response associated with a dense inflammatory infiltrate and some loss of normal architecture. Significant immune infiltration also was observed with injection of Ad-VSV-G alone and, in addition, syncytial-like structures were observed in injected prostates, consistent with the fusogenic activity of the VSV-G protein. Intraprostatic injection of Ad-VSV-G and Ad-hsp70 caused severe infiltration, necrosis, and tissue destruction consistent with results from intradermal injection of plasmids expressing HSVtk and hsp70 (Daniels et al., supra; and Sanchez-Perez et al. (2005), supra). Unlike those experiments, however, the dense infiltration with immune cells was persistently present in prostate tissue and did not significantly resolve up to 3 weeks postinjection. This persistent inflammation was associated with an ongoing autoimmune destruction of the prostate as reflected by a progressive decrease of the wet weight of prostates recovered from treated animals (P<0.01 for Ad-GFP and Ad-VSV-G+Ad-hsp70).

Hsp70 Induces IL-6 from Prostate Tissue

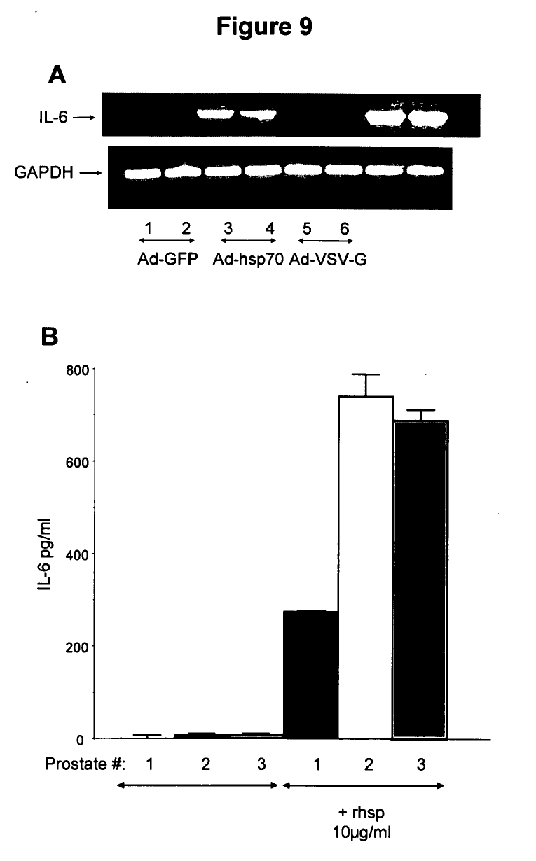

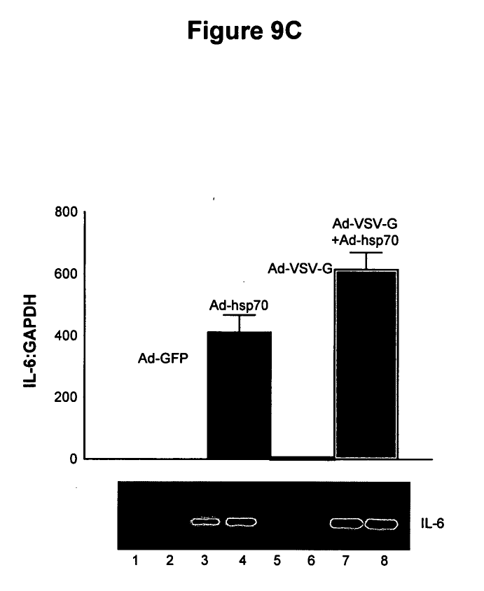

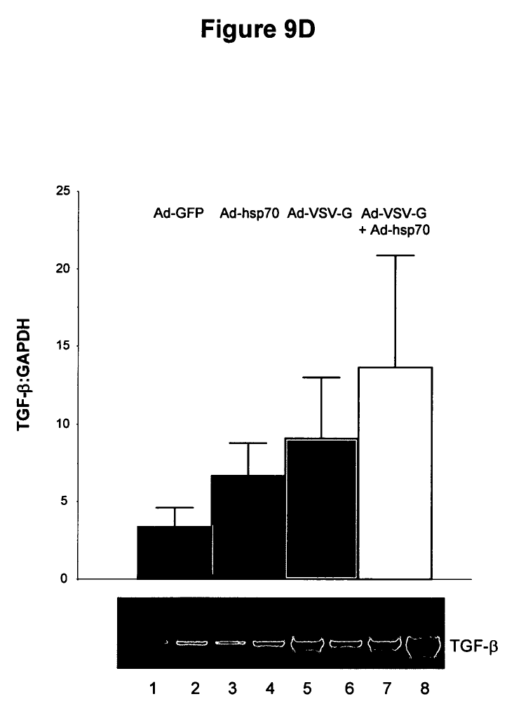

A screen of injected prostates by reverse transcriptase PCR (RT-PCR) for different cytokines indicated that IL-6 was consistently induced in prostates injected with Ad-hsp70 (whether or not Ad-VSV-G or Ad-GFP were also injected; FIG. 9A). These results were confirmed at the protein level by treating explanted and dissociated prostate with recombinant hsp70 (FIG. 9B; P<0.001 for all three prostates tested when compared plus or minus hsp70). The lymph nodes draining the injected prostates also were assayed to investigate the profile of cytokine expression induced by local inflammatory killing, which will directly influence the outcome of T-cell priming Lymph node draining the injected prostates again showed IL-6 expression in mice injected with the Ad-hsp70 vector (but not in mice injected with other adenovirus vectors, indicating that IL-6 is not a response to the adenovirus per se; FIG. 9B). Importantly, TGF-β was expressed in the majority of the lymph node, largely irrespective of the adenovirus vectors that were injected into the associated organs.

### Lymph Nodes Draining the Prostates Undergoing Inflammatory Killing Contain IL-17

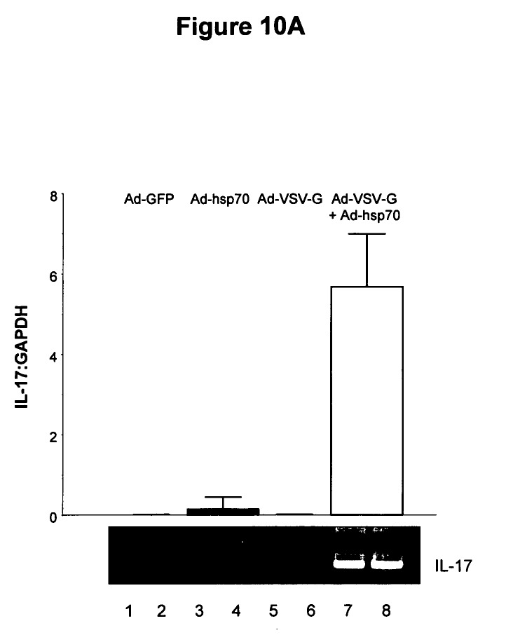

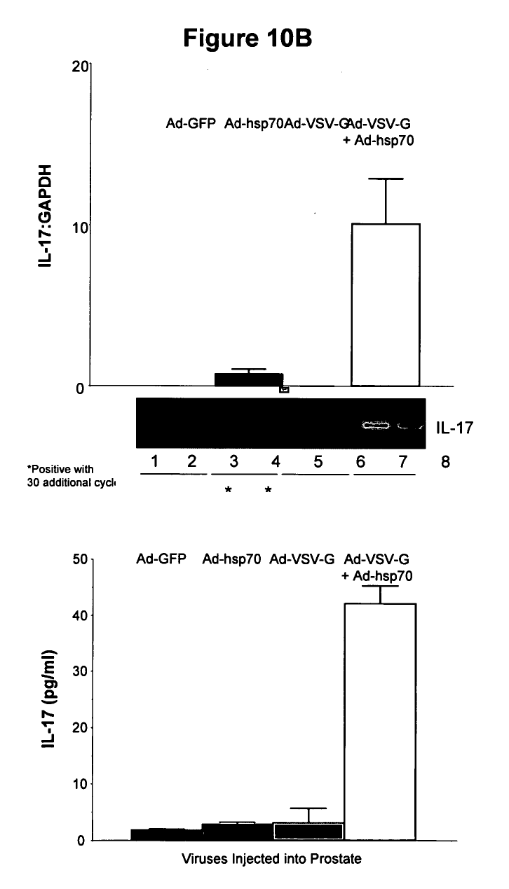

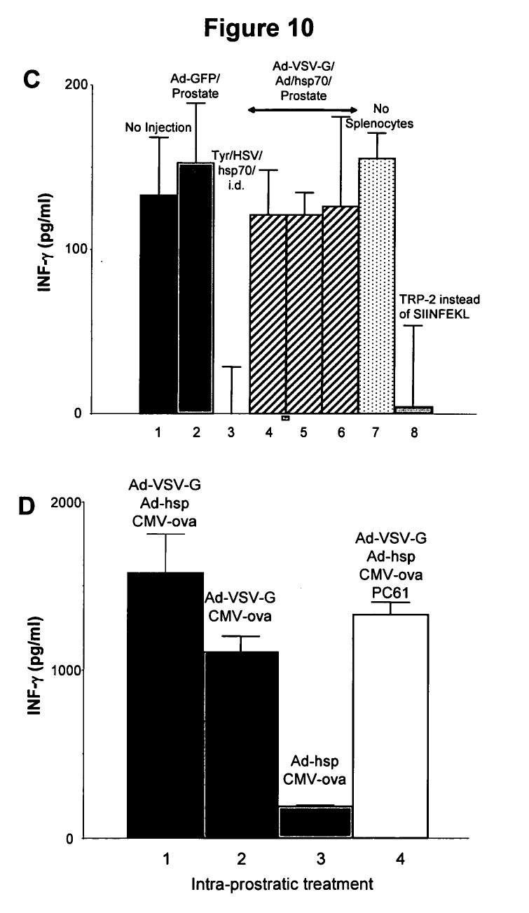

The ongoing autoimmune inflammation and destruction of the prostate, combined with detection of both IL-6 and TGF-β in the lymph node draining the injected prostates, suggested that Ad-VSV-G+Ad-hsp70 treatment of prostate may generate progressive autoimmunity through induction of a Th-17 response, differentiation of which is characterized by a combination of TGF-β and IL-6 (Veldhoen et al., Immunity, 24:179-189 (2006); Mangan et al., Nature, 441:231-234 (2006); and Bettelli et al., Nature, 441:235-238 (2006)). Consistent with this hypothesis, mRNA for IL-17 was detected in both prostates injected with Ad-VSV-G+Ad-hsp70 (FIG. 10A) and also in the draining lymph node (FIG. 10B). This result was confirmed at the protein level in lymph node (FIG. 10B; P<0.001 for treatment with Ad-VSV-G+Ad-hsp70 compared with all the other three treatments).

Studies were then conducted to test for generation of Treg responses. Splenocytes recovered from normal mice (uninjected) cannot significantly suppress IFN-γ secretion from activated T cells in the presence of their cognate antigen (FIG. 10C, lanes 1 and 7; P>0.05). Splenocytes from mice undergoing inflammatory killing of normal melanocytes, however, contain suppressor activity associated with the generation of Treg and TGF-β (Daniels et al., supra; and Sanchez-Perez et al. (2005), supra); FIG. 10C, lanes 3 and 7; P<0.002 (Tyr-HSV/hsp70/i.d.)]. In contrast, splenocytes from mice injected intraprostatically with Ad-VSV-G+Ad-hsp70 were unable to exert any suppression of activated T cells in this assay (as represented by the positive control of OT-1 cells alone; lane 7) even when spleens were harvested at different times after prostatic injection (FIG. 10C, lanes 4-7; P>0.05), suggesting that inflammatory killing of normal prostates does not induce significant Treg responses.

The repertoires of known tissue/tumor-associated antigens in prostate cancer are much less well-characterized than for the melanoma model. Therefore, the ova antigen was used as a model to characterize how inflammatory killing of normal cells affects the generation of antigen-specific responses. When a plasmid expressing the ova protein was coinjected into the prostates of C57Bl/6 mice along with different adenoviral treatments, both Ad-VSV-G (P<0.02 compared with Ad-hsp70/CMV-ova) and, more potently, Ad-VSV-G+Ad-hsp70 (P<0.01) primed easily detectable anti-ova responses in splenocytes from those mice (FIG. 10D). This anti-ova reactivity was neither enhanced nor diminished in mice in which Treg had previously been depleted by antibody treatment before viral injections (no significant difference between lanes 1 and 4).

Taken together, these data indicate that inflammatory killing of normal prostates does not induce a significant Treg response.

Loss of IL-6 Converts a Th17 Response into a Treg Response In Vivo

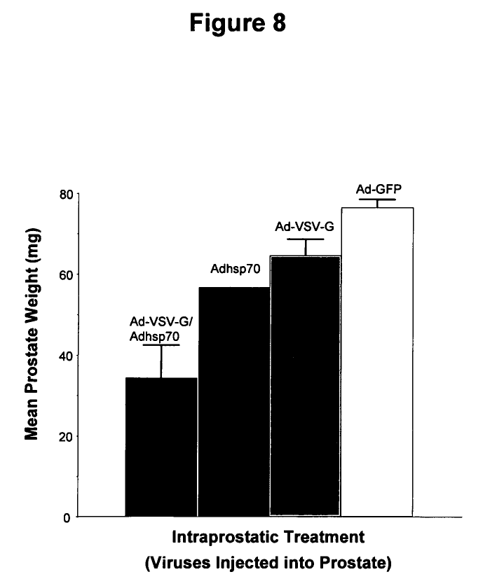

The above data suggest that the IL-6 response of prostate tissue to hsp70 expression drives the resultant immune response against tissue-associated self-antigens down a Th17 pathway. To test the central importance of IL-6, several of these experiments were repeated in IL-6KO mice. Whereas injection of Ad-VSV-G+Ad-hsp70 into the prostates of C57Bl/6 mice led to progressive chronic destruction of the prostates associated with intense immune infiltration, no significant damage or infiltration was observed in similarly injected prostates of IL-6KO mice, and there was no difference between injection of Ad-VSV-G+Ad-hsp70 or Ad-GFP. Similarly, there was no significant difference between the wet weights of prostates of IL-6KO mice injected with either Ad-VSV-G+Ad-hsp70, Ad-GFP, or PBS 60 days after viral injection—in contrast to the reduction in prostate weights of up to 50% seen in C57Bl/6 mice (FIG. 8).

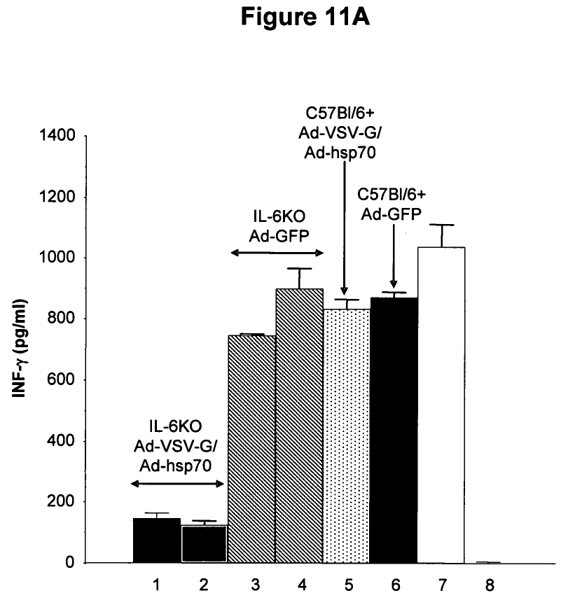

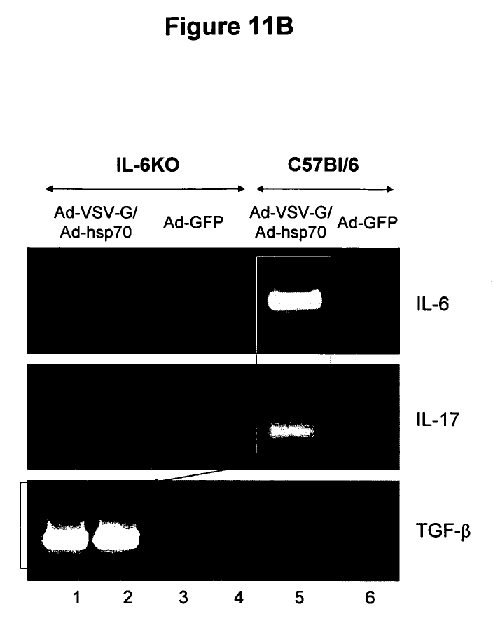

There was a dramatic difference, however, in the ability of splenocytes from IL-6KO mice injected intraprostatically with Ad-VSV-G+Ad-hsp70 to suppress IFN-γ secretion from activated T cells. Whereas splenocytes from Ad-VSV-G+Ad-hsp70-injected C57Bl/6 mice contained no detectable suppressive activity in this assay (FIG. 10C), splenocytes from IL-6KO mice were potently inhibitory to activated T cells when the prostates had undergone inflammatory killing with Ad-VSV-G+Ad-hsp70—but not with intraprostatic injection of Ad-GFP (FIG. 11A, lanes 1 and 2 compared with lanes 3 and 4; P<0.01 in all cases). The mechanism of this suppression was shown to be dependent in large part on TGF-β. Thus, when splenocytes from IL-6KO mice treated intraprostatically with Ad-VSV-G+Ad-hsp70 were cocultured with activated OT-1 T cells, IFN-γ production from the OT-1 T cells was significantly inhibited as shown in FIG. 11A. These splenocytes also were cocultured with activated OT-1 in the presence of 50 ng/mL of 341-BR TGF-β sRII/Fc (R&D Systems), a 159 amino acid extracellular domain of human TGF-β receptor type II fused to the Fc region of human IgG1, to neutralize TGF-β. In two such experiments, 341-BR TGF-β sRII/Fc increased the amount of IFN-γ secreted by activated OT-1 and in the presence of splenocytes from IL-6KO mice treated with Ad-VSV-G+Ad-hsp70, by about 5- to 6-fold (mean values of 130 pg/mL in the absence of 341-BR TGF-3 sRII/Fc to 710 pg/mL in its presence) —approaching the levels of IFN-γ produced by splenocytes of IL-6KO mice injected with Ad-GFP as the negative control (815 pg/mL). These data indicate that the suppressive effects of splenocytes from IL-6KO mice on activated T cells is mediated, in part at least, by TGF-β.

To confirm the transition of the immune response to inflammatory killing from a Th-17 autoimmune response to Treg protective immunity in these IL-6KO mice, the microenvironment of the injected prostates was examined As before, prostates of C57Bl/6 mice injected with Ad-VSV-G+Ad-hsp70 contained readily detectable levels of IL-6 and IL-17 but only minimal TGF-β (FIG. 11B). In the absence of IL-6, however, no IL-17 was detected in injected prostates and these organs now contained abundant TGF-β, indicative of a much more immunosuppressive tissue microenvironment. These data show that by removing IL-6, the prostate-specific IL-6/Th-17 immune response to inflammatory killing of normal cells was converted into a Treg response.

Prostate Autoimmunity Correlates Closely with Tumor Rejection

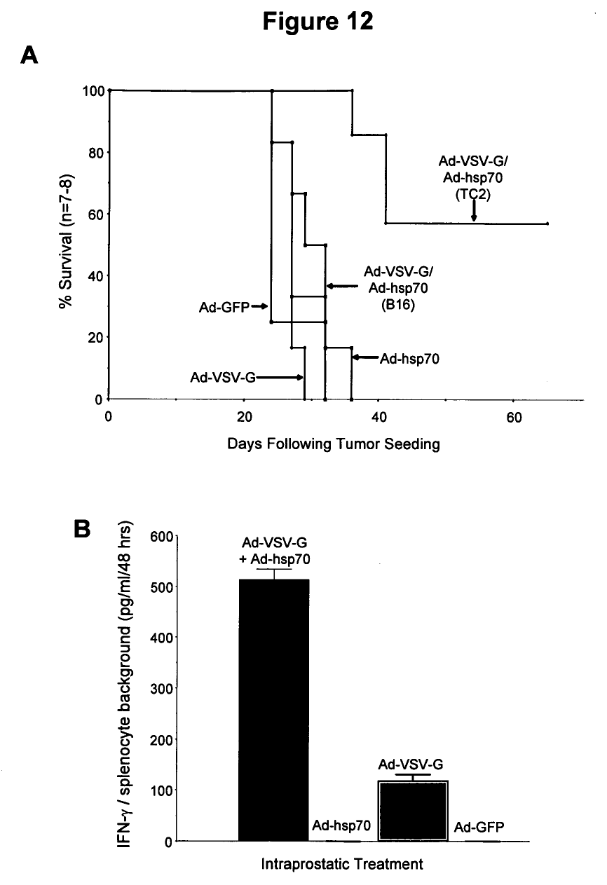

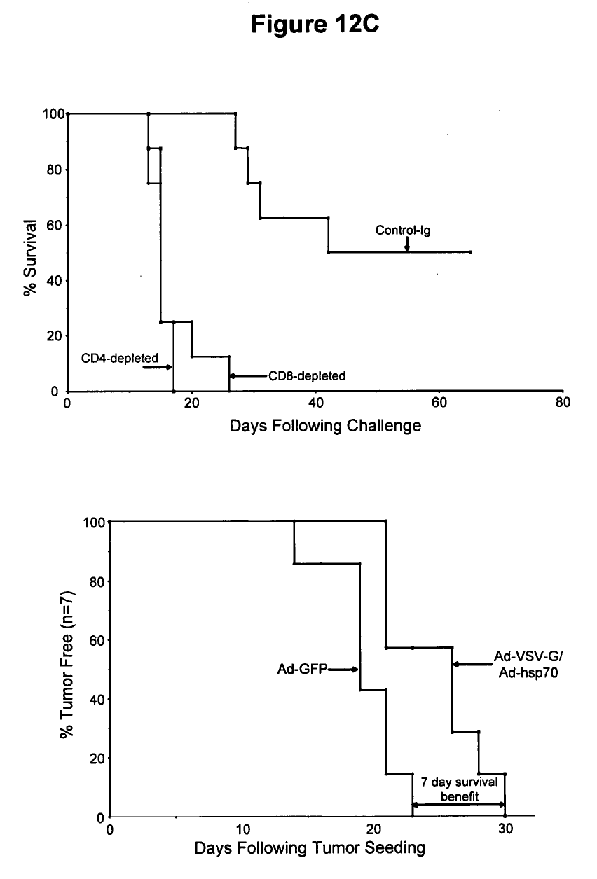

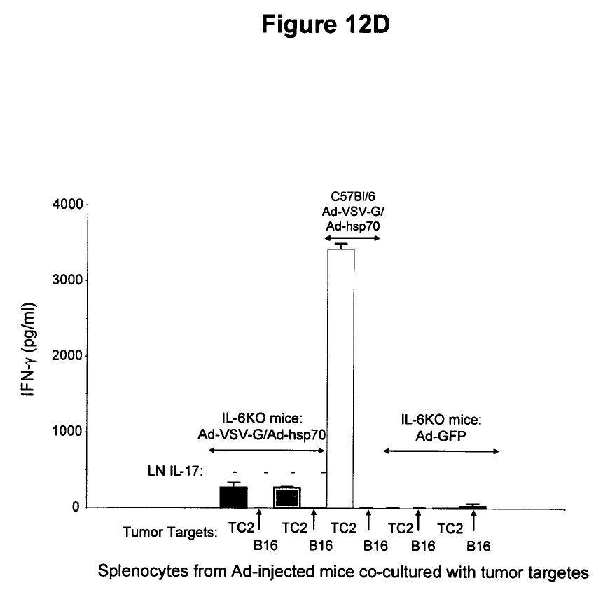

Of particular interest, as described herein, was whether the autoimmune response induced by inflammatory killing of normal cells can be exploited to treat tumors of the same histologic type sharing common antigens with the tumor. TC2 cells are murine prostatic cancer cells syngeneic to C57Bl/6 mice. Direct intraprostatic injection of control adenoviruses into animals bearing 6 days established TC2 tumors growing s.c. was unable to affect the growth of the tumors (FIG. 12A; P>0.05 for Ad-VSV-G compared with Ad-hsp70 or Ad-GFP). The combination of Ad-VSV-G+Ad-hsp70, however, induced a potent tumor rejection response, which cured between 50% and 80% of mice depending on the experiment (FIG. 12A; P<0.001 for Ad-VSV-G+Ad-hsp70 compared with Ad-VSV-G; Ad-hsp70 or Ad-GFP alone). This rejection response was highly prostate specific, as mice treated in the same way with Ad-VSV-G+Ad-hsp70 were unable to reject s.c. B16 melanoma tumors. Moreover, animals that rejected the primary TC2 tumors also were protected against rechallenge with a tumorigenic dose of TC2 cells 70 days after the initial challenge. Consistent with both the inability to reject nonprostate-derived tumors and the generation of long term immunologic memory, splenocytes from mice treated with Ad-VSV-G+Ad-hsp70 contained cells specific for prostate antigens expressed on TC2 cells (FIG. 12B) but not for antigens expressed on B16 cells. The tumor rejection response induced by inflammatory killing of normal prostate was dependent on both CD8⁺ and CD4⁺ cells (FIG. 12C, top; P=0.001 for control immunoglobulin-treated group compared with either CD4⁺ or CD8⁺ T-cell-depleted groups). These results contrast to those in the melanocyte/melanoma model where CD4⁺ T cells were dispensable for therapeutic effects [Ferrone, Nat. Biotechnol. 22:1096-1098 (2004); Sanchez-Perez, (2006), supra; Daniels et al., supra; and Sanchez-Perez et al., (2005), supra), but are consistent with the role of CD4⁺ Th17 cells in driving both the autoimmune and antitumor immune responses that we observe in this system (Veldhoen et al., supra; Mangan et al., supra; and Bettelli et al., supra). Consistent also with the observation that IL-6 is a critical mediator of the differentiation of a Th-17 antiprostate autoimmune response (FIG. 11), TC2 tumors were not cured by inflammatory killing of normal prostates in IL-6KO mice (FIG. 12C, bottom), unlike the result in C57Bl/6 mice (FIG. 12A). There was, however, a small but significant (P<0.02) prolongation of survival between groups injected with Ad-GFP and Ad-VSV-G+Ad-hsp70, suggesting that factors other than just IL-6 also may be important after hsp70-mediated immune activation in the prostate. This also is consistent with the observation that splenocytes from IL-6KO mice, treated with intraprostatic injections of Ad-VSV-G+Ad-hsp70, secreted IFN-γ in response to coculture with TC2 tumor cells (but not B16 cells), but at greatly reduced levels than was the case for splenocytes from C57Bl/6 mice (FIG. 12D). Finally, IL-17 was undetectable by RT-PCR and ELISA from lymph nodes draining the injected prostates in IL-6KO mice, which was not the case for C57Bl/6 mice (FIG. 12D, FIG. 10).

Taken together, the results presented in this example demonstrate that inflammatory killing of normal prostate was highly effective at curing established metastatic prostatic tumors but not tumors of a different histologic type. These results are significant in at least the following respects. First, they show that autoimmune disease of the prostate can be induced by specific cytokine responses to one, or a few, key pathogenic-like signals. Second, they show that the intimate connectivity between autoimmune and antitumor rejection responses extends beyond the classic melanoma paradigm. In addition, they suggest that the principle of inflammatory killing of normal cells to treat neoplastic disease is applicable to tumors other than just melanoma.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated nucleic acid comprising:
(a) a sequence encoding a CD40L polypeptide, a sequence encoding a chaperone polypeptide, and a sequence encoding a cytotoxic polypeptide;
(b) a sequence encoding a CD40L polypeptide and a sequence encoding chaperone polypeptide; or
(c) a sequence encoding a CD40L polypeptide and a sequence encoding a cytotoxic polypeptide.

2. The nucleic acid of claim 1, wherein said CD40L polypeptide is a human CD40L polypeptide.

3. The nucleic acid of claim 1, wherein said chaperone polypeptide is a human hsp70 polypeptide.

4. The nucleic acid of claim 1, wherein said cytotoxic polypeptide is a herpes simplex virus thymidine kinase polypeptide.

5. The nucleic acid of claim 1, wherein said cytotoxic polypeptide is a fusogenic membrane G glycoprotein of vesicular stomatitis virus.

6. The nucleic acid of claim 1, wherein said nucleic acid is a plasmid.

7. The nucleic acid of claim 1, wherein said nucleic acid is a viral vector.

8. A composition comprising:
(a) a nucleic acid molecule encoding a CD40L polypeptide, a nucleic acid molecule encoding a chaperone polypeptide, and a nucleic acid molecule encoding a cytotoxic polypeptide;
(b) a nucleic acid molecule encoding a CD40L polypeptide and a nucleic acid molecule encoding a chaperone polypeptide; or
(c) a nucleic acid molecule encoding a CD40L polypeptide or a nucleic acid molecule encoding a cytotoxic polypeptide.

9. The composition of claim 8, wherein said CD40L polypeptide is a human CD40L polypeptide.

10. The composition of claim 8, wherein said chaperone polypeptide is a human hsp70 polypeptide.

11. The composition of claim 8, wherein said cytotoxic polypeptide is a herpes simplex virus thymidine kinase polypeptide.

12. The composition of claim 8, wherein said cytotoxic polypeptide is a fusogenic membrane G glycoprotein of vesicular stomatitis virus.

13. The composition of claim 8, wherein one or more of said nucleic acid molecules is a plasmid.

14. The composition of claim 8, wherein one or more of said nucleic acid molecules is a viral vector.

15. A method for inducing immunity against cancer, wherein said method comprises administering nucleic acid encoding a CD40L polypeptide, a chaperone polypeptide, and a cytotoxic polypeptide to a mammal having said cancer under conditions wherein said CD40L polypeptide, said chaperone polypeptide, and said cytotoxic polypeptide are expressed.

16. The method of claim 15, wherein said mammal is a human.

17. The method of claim 15, wherein said cancer is a melanoma cancer.

18. The method of claim 15, wherein said cancer is a prostatic cancer.

19. The method of claim 15, wherein said nucleic acid is a single nucleic acid encoding said CD40L polypeptide, said chaperone polypeptide, and said cytotoxic polypeptide.


SEQUENCE LISTING (3 sequences)

SEQ ID NO: 1 - protein, 9 aa, Artificial Sequence
  Synthetic
KVPRNQDWL

SEQ ID NO: 2 - protein, 9 aa, Artificial Sequence
  Synthetic
SVYDFFVWL

SEQ ID NO: 3 - protein, 8 aa, Artificial Sequence
  Synthetic
SIINFEKL